(19) United States
(12) Patent Application Publication
(10) Pub. No.: US 2026/0355451 A1
(43) Pub. Date: Nov. 12, 2026

(54) BEDSIDE SLEEP ASSISTANT SYSTEM TO DIFFERENTIATE SLEEPING PATTERNS FOR MULTIPLE USERS

(71) Applicant: Ambient Life Inc., Newton, MA (US)

(72) Inventors: Adrian Canoso, Newton, MA (US); Stephan Wirth, Sóller (ES)

(21) Appl. No.: 19/435,280

(22) Filed: Dec. 29, 2025

Related U.S. Application Data
(63) Continuation of application No. PCT/US24/37009, filed on Jul. 8, 2024.
(60) Provisional application No. 63/512,202, filed on Jul. 6, 2023.

(51) Int. Cl. A61M 21/00

(52) U.S. Cl. CPC A61M 21/00; A61M 2021/0027; A61M 2021/0044; A61M 2205/502; A61M 2230/005; A61M 2230/40; A61M 2230/63

(57) ABSTRACT

A system may include a device and a second device that may include a lighting system and audio system integrated with a housing, a user interface for receiving user commands, a monitoring sensor system for monitoring a sleep state, and a communication system. The monitoring sensor system may include a radar system, microphone, and have an operating area including a user sleep area. A controller may be configured to receive sleep data from the monitoring sensor system, and execute a sleep assistance function that performs a sleep assist output function based on the received sleep data.

### CROSS REFERENCE TO RELATED APPLICATIONS

This application claims benefit of and is a continuation of International Patent Application No. PCT/US2024/037009 (Attorney Docket No. 2100.0008), filed Jul. 8, 2024, and entitled “BEDSIDE SLEEP ASSISTANT,” International Pub. No. WO 2025/010441, which is hereby incorporated by reference in its entirety for all purposes.

International Patent Application No. PCT/US2024/037009 claims priority to U.S. Provisional Patent Application No. 63/512,202, filed on 6 Jul. 2023, entitled “BEDSIDE SLEEP ASSISTANT” (Attorney Docket No. 2100.0002).

The foregoing application is incorporated herein by reference in the entirety for all purposes.

### BACKGROUND

Described herein are methods and systems to provide improved sleep management, such as for helping people that suffer from stress-induced insomnia, a problem that affects over 70 million people in the US. The design approach is founded on key tactics based on cognitive behavioral therapy for insomnia (CBT-I) and sleep hygiene best practices to create a holistic solution to help people sleep better.

### SUMMARY

The bedside sleep assistant is supportive of a sleep experience that minimizes cognitive load to avoid complexity, frustration, or prolonging wakefulness as commonly experienced with mobile devices and social media apps.

In embodiments, the techniques described herein relate to a bedside sleep assistant device, including: a housing; a lighting system integrated with the housing; an audio system integrated with the housing; a user interface for receiving user commands; a monitoring sensor system for monitoring a sleep state, wherein the monitoring sensor system has an operating area including a user sleep area; and a controller including a processor and a memory, the controller configured to store a set of instructions that, when executed, cause the controller to: receive sleep data from the monitoring sensor system; and execute a sleep assistance function that performs a sleep assist output function based on the received sleep data.

In embodiments, the techniques described herein relate to a lighting system, including: a user interface configured to receive a position input; a directable lighting source, wherein an illumination direction of the directable lighting source is electronically directable; and a controller including a processor and a memory, the controller configured to store a set of instructions that, when executed, cause the controller to: receive the position input from the user interface; and direct the illumination direction of the directable lighting source to correspond to the received position input from the user interface.

In embodiments, the techniques described herein relate to a bedside sleep assistant device, including: a housing; a lighting system integrated with the housing; an audio system 1806 integrated with the housing; a user interface for receiving user commands; a monitoring sensor system for monitoring a sleep state, wherein the monitoring sensor system has an operating area including a user sleep area; and a controller including a processor and a memory, the controller configured to store a set of instructions that, when executed, cause the controller to: receive a plurality of sleep data from the monitoring sensor system over a period of time; determine a measured sleep pattern value for a user present in the user sleep area based on the received plurality of sleep data, the measured sleep pattern value a measure of sleep quality; compare the measured sleep pattern value to a stored nominal sleep pattern value, the stored nominal sleep pattern value a reference measure of a healthy sleep quality; if a difference between the measured sleep pattern value and the stored nominal sleep value exceeds a threshold, then provide at least one recommended action to concentrate a sleep period of the user; and receive a second plurality of sleep data over a second period of time, wherein the process to determine and compare is repeated until the difference between the measured sleep pattern value and the stored nominal sleep pattern is changed.

These and other systems, methods, objects, features, and advantages of the present disclosure will be apparent to those skilled in the art from the following detailed description. The example benefits are non-limiting, and a given embodiment may lack one or more, or all, of the described benefits, and/or may include additional benefits that are not set forth in this summary.

All documents mentioned herein are hereby incorporated in their entirety by reference.

References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE FIGURES

The disclosure and the following detailed description of certain embodiments thereof may be understood by reference to the following figures:

FIGS. 1A-1D are embodiment diagrams of example hardware design, features, and interaction points of the bedside sleep assistant.

FIGS. 2A-2B are embodiment diagrams of an example home screen interface on the display of the bedside sleep assistant.

FIG. 3 is an embodiment diagram of example depiction of the bedside sleep assistant as a central hub for sleep & what it integrates with.

FIGS. 4A-4B are embodiment diagrams with example application user settings for sleep routines and wake up schedules.

FIGS. 5A-5C are embodiment diagrams of example light direction adjustment feature.

FIGS. 6A-6B are embodiment diagrams of example brightness adjustment and focus lighting control.

FIGS. 7A-7C are embodiment diagrams of example journaling via the bedside sleep assistant or through the bedside sleep assistant's mobile application.

FIG. 8 is an embodiment diagram of example journaling via the bedside sleep assistant or through the bedside sleep assistant's mobile application.

FIG. 9 is an embodiment diagram depicting creating and playing back a sound journey.

FIG. 10 is an embodiment diagram depicting content selection being made from one track to the other.

FIG. 11 depicts an embodiment sleep restriction process sequence with optional paired device.

FIG. 12 depicts an embodiment radar based night light process sequence with optional paired device

FIG. 13 depicts an embodiment combination of a scene.

FIG. 14 depicts an embodiment scene flow sequence.

FIG. 15 depicts an embodiment personalized artificial intelligence (AI) generated sleep story creation and delivery process sequence.

FIG. 16 depicts an embodiment diagram for the bedside sleeping assistant device.

FIG. 17 depicts an embodiment diagram for a directed lighting system.

FIG. 18 depicts an embodiment diagram for a sleep restriction device.

### DETAILED DESCRIPTION

The bedside sleep assistant may combine lighting, sound, sensors, such as environmental sensors, tactile controls, touch displays, radar, cameras, and the like. The bedside sleep assistant may be internet connected, such as with the ability to integrate with APIs, such as health APIs, IoT protocols, online services to stream content, and the like. The bedside sleep assistant, as described herein, may also be referred to as a sleep assistant, a bedside sleep assistant device, or simply as a device, in the context of describing embodiments of the bedside sleep assistant.

In aspects, simple touch or motion gestures like swiping down on the screen or in the air may initiate sleep content. Physical controls may precisely adjust functions, such as volume, dimmer slider dials in the right light levels, and the like, all packaged in a compact, approachable form factor.

The bedside sleep assistant may provide benefits to individuals or couples trying to sleep better, such as by providing stress management, for example, to help mitigate cognitive hyperactivity with instant access to proven sleep interventions like breathing techniques, body scans, sleep stories, automated content delivery, and the like. The bedside sleep assistant may integrate lighting cues to help users pace breathing and relaxation techniques, and provide directed lighting to adapt to varying nighttime lighting needs. Another benefit of the bedside sleep assistant may include sleep routine support, such as through configuration of sleep duration, bedtime, wake-up schedules, and the like, that help gently cue the user when it's time for bed and time to wake up. The bedside sleep assistant may enable easy access to temporary alarm changes to protect the user's sleep schedule. Moreover, the device may offer contactless sleep tracking, delivering sleep reports (i.e., sleep duration, sleep stage duration, contiguity, regularity) and personalized recommendations to further enhance sleep quality. Another benefit of the bedside sleep assistant may include improved sleep hygiene, environmental monitoring of light, temperature, and humidity, and the like, to help keep track of ideal sleep conditions.

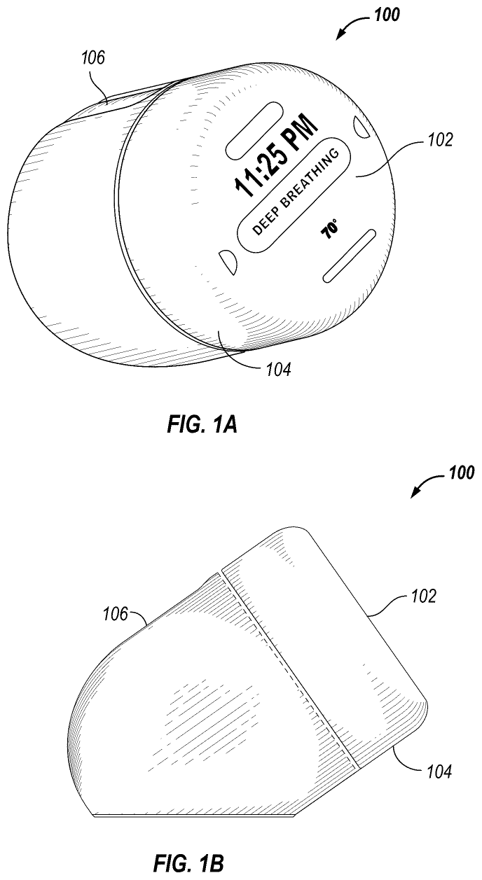

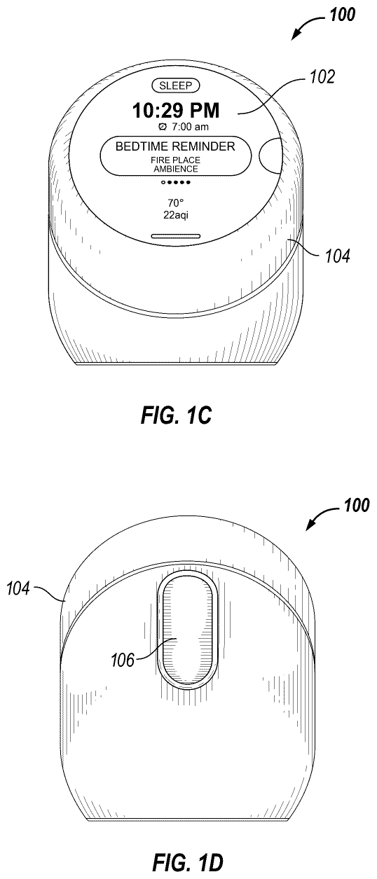

Referring to FIGS. 1A-1D, an embodiment of a bedside sleep assistant 100 is depicted. In an aspect, the layout of the bedside sleep assistant's controls may include direct access to a precision control ring 104, a capacitive touch strip 106, and the like. For instance, a control ring 104 and a capacitive touch strip 106 may be unique to each other to make them easy to discern and interact with without the need for line of sight. In embodiments, the control ring may encompass a touchscreen display 102.

In embodiments, a control ring 104 may be located at the front of the bedside sleep assistant and encompass the display 102. It may operate as a dial, such as used for input modalities, for instance, as volume control, setting time, scrolling lists, and the like.

The bedside sleep assistant's control ring 104 may function as a lens for the lighting system. The lighting system may be designed to provide low-intensity light directly at the user and diffused light around the bedside sleep assistant. Traditionally, wake-up lights project light directly at the user, which can be visually uncomfortable when awakening. Additionally traditional lights are controlled by PWM only, which limits the amount of dimming before flickering becomes noticeable. Additionally, typical lighting systems have coarse adjustment. The bedside sleep assistant lighting system may be able to reproduce light output that can become as dim as the human eye can perceive and as bright as a reading light. The device's lighting system may consists of RGB (Color) and white LEDs that are controlled with both PWM (pulse width modulation) and current to enable a wide dynamic range from low light output to maximum light output. The light output for the LEDs may be controlled by a LED controller and SoC (system on chip). White LEDs emit a warm light, and each LED element is individually controllable by the onboard controller. The LEDs may project light through a segmented lightpipe that diffuses and disburses the LED light both in the direction of the display and radially around the translucent control ring. This is so light can be provided as both direct and indirect light in one system. The lighting system may be positioned to the user so light can be emitted directionally from the front, back, and sides of the device, allowing for direct and diffuse lighting needs.

In embodiments, a capacitive touch strip 106 may operate as a rotary dial and button. The touch strip 106 may be oriented along the “spine” of the bedside sleep assistant to offer easy access as a user interface. Interactions like sliding and tapping may be used to adjust the light, toggle between lights, select menu options, and the like.

The physical controls of the bedside sleep assistant may have benefits with respect to traditional similar devices. For instance, typically, similar devices have comparatively small buttons whose functionality is difficult to differentiate in a dimly lit setting. Traditional lighting systems often consist of table lamps that rarely have a dimmer switch. Typically, when brightness and volume settings are adjusted on devices, they have coarse increments making it difficult to make fine-grained adjustments, especially at low volume or low brightness settings.

In contrast, input modalities for the bedside sleep assistant may provide an improved user interface through controls that are asymmetric and unique, creating highly differentiated interaction points to facilitate operation in lit and dark environments.

Control input elements may be positioned close together, such as to be interacted with using just one finger. Additionally, the Sleep assistant may not require an external mobile application to configure the device, which minimizes the use of distracting devices.

### Sightless Interactions

Traditionally, sleep aids like sound machines and alarm clocks require line-of-sight to operate via dials and/or buttons to change settings like alarms, auto-off timers, sound selection, and volume adjustment. These interaction points typically require a close line of sight and light when operated in the dark. Additionally, in some cases, traditional buttons may emit a sound when pressed (such as a clicking sound), which may be disruptive for users that share the room with a partner. In solutions such as with a typical mobile device, interacting with sleep content in an application requires a high cognitive load to pick up, unlock, and navigate to the application, followed by interacting with the application itself for managing sleep-related content. This series of interactions can awaken the user, often leading them to use social media and other apps. Traditionally, voice-based smart speakers typically have little to no tactile interfaces, requiring the user to speak to the device. Voice interactions and responses may be disruptive for users that share the room with a partner. Typical smart displays have interfaces that require a series of interactions like navigating menus and pressing on-screen buttons to access apps and content. As with mobile devices, the cognitive load needed for these interactions may awaken the user.

The bedside sleep assistant's interface is specifically designed for an improved user experience. This may be particularly advantageous for users who require glasses to see properly, are visually impaired, are experiencing visual disabilities, are falling asleep, are struggling with low dexterity, are in a state of drowsiness, wearing a sleep mask, and the like. This improved user experience may be achieved through singular or combination of silent tactile controls, a touchscreen display, a radar, and the like, such as to enable touch, tactile, touchless, and the like control of functions. For instance, the user's presence, sleep state, hand motions or gestures across the touch screen or in view of an integrated radar may provide control of a bedside sleep assistant's function.

For example, the radar may detect the user's presence in the room and/or sleep state changes (e.g., awake, light sleep, or deep sleep) and adapt a sleep assistance function accordingly (e.g. change scene, change volume, or change lights when user falls asleep). Another example may be the ability for the user to hide a displayable clock through a preference setting, where tapping on the screen may temporarily re-display the clock to “peek” at the time.

The bedside sleep assistant may provide for an improved user experience due to a sleep routine, an overall sleep program that delivers sleep interventions, noise masking, and wake up behaviors for the user. The lighting, video, and audio content that comprise the sleep routine are referred to herein as scenes.

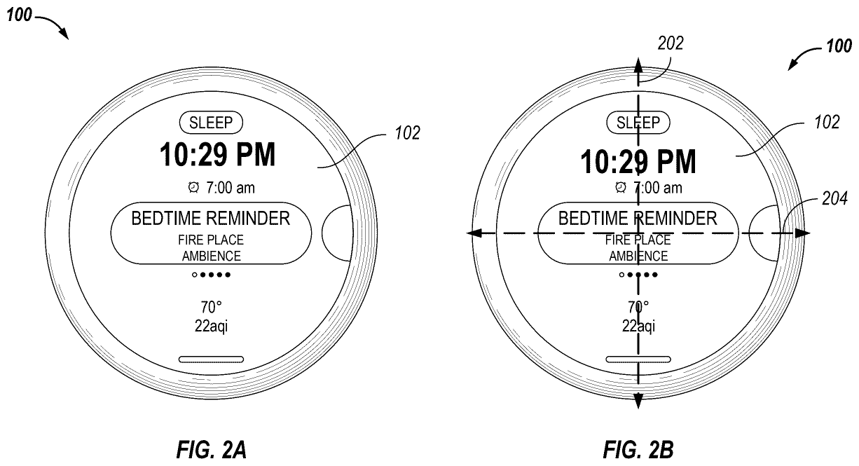

For instance, and referring to FIGS. 1A-1D and 2A-2B, in a non-limiting example, the display 102 may be a touch display, with touch movements (e.g., swiping or dragging) in any combination of movements across the X-axis 204 and Y-axis 202. For instance, to start the sleep routine, the user may perform a single downward swipe anywhere on the bedside sleep assistant's touchscreen display. The user interface elements help communicate what scene is “loaded” and a “next scene indicator” showing what scene will come next, such as with a downward swipe (see FIGS. 2A-2B).

Conversely, swiping up may return to the previous scene. To change content, the user may swipe left or right anywhere on the display. To adjust volume, the user may rotate the control ring (see FIGS. 1A-1D). To adjust brightness, the user my interact with the brightness strip, such as with a swipe up or down to adjust brightness or turn the light on or off (see FIGS. 1A-1D). To set a snooze while the bedside sleep assistant is in the wake-up alarm scene, the display may show a “snooze” button, where a swipe down may stop the alarm. Each of these adjustments outlined may be automatically saved to preserve the user's preferences.

### Automated Scenes and Sleep Routines

Traditionally, content is statically presented to the user with sleep aids such as sound machines, through mobile device apps, and the like. Mobile devices usually have a user configurable shut-off timer for content apps and a separate app for the alarm. Typically, the user must take several steps to unlock, navigate, start a relaxation app, and configure a sleep timer. This multi-step process has a high cognitive load and can cause wakefulness and at times, the urge to use other apps such as email, social media and the like, prolonging the amount of time it takes the user to fall back asleep.

The implementation of automated scenes may enable the user to use one gesture to start a sequence called a Sleep Routine that may include content delivery, sleep interventions, interaction with similar paired sleep assistants, IoT connected devices such as automatic blinds, lights, thermostat, and the like, thus enabling them to fall asleep more easily by reducing cognitive load and distraction when compared to mobile device use.

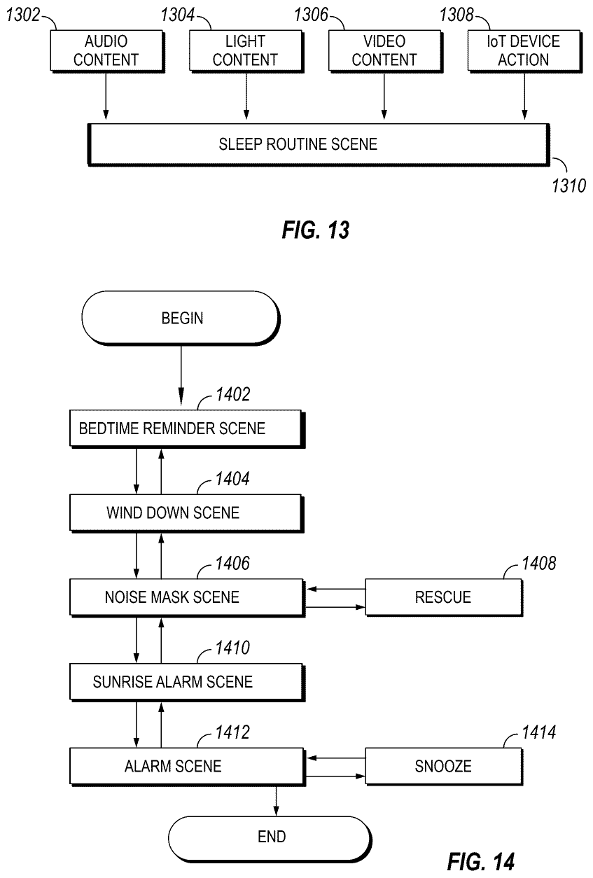

The bedside sleep assistant may provide a sleep routine (aka Sleep assistance function) which is comprised of an automated set of scenes that carry the user through phases of the sleep experience. For instance, and referring to FIG. 13, composition of a sleep routing scene 1310 may include audio content 1302, light content 1304, video content 1306, IoT device action 1308, and the like. Scenes can combine visual and auditory content delivered through the device's audio system, lighting, display, and IoT connected devices. As such, the bedside sleep assistant may unify the delivery of the right sleep interventions and content (or periods of no content) from the start of sleep through waking up in the morning. The bedside sleep assistant may string together scenes triggered through a variety of approaches based on user inputs, biometric sleep sensing, and external sensors and similar paired devices.

In embodiments, a scene may be any combination of lighting, video, imagery, audio content, IoT connected device (such as IoT lightbulbs, IoT curtains, IoT outlets, etc.) and the like. Scenes within the sleep routine can be comprised of audio, video, and lighting effects consisting of but not limited to local content played from the device, podcasts, generative content, streaming content via API integrations enabling the user to access content from other service providers, audio books, and the like.

Scenes may also trigger external actions through API calls to control other wirelessly or USB connected devices such as thermostats, temperature controlled mattresses, lighting systems, and the like. For instance, in a non-limiting example, a user can access their Meditation App to access their favorite content to be played back on the bedside sleep assistant. Additionally, while the sleep routine is active, the user can add their temperature-controlled mattress to the sleep assistant, which enables the user to command the temperature controlled mattress when to turn on, adjust, and turn off the temperature control feature of the mattress through the sleep assistant's user interface.

Automated behaviors may deliver a seamless sleep experience to help cue users when it's time for sleep or wind down, such as including changes to lighting and/or audio, altering display presentation (e.g., dimming or turning off), and the like. Once the user is asleep, the bedside sleep assistant may provide optional noise masking, optional sleep interventions, automatically transition to a wake-up routine, and the like. In embodiments, and referring to FIG. 14, the bedside sleep assistant 100 may transition between a plurality of scenes, such as bedtime reminder scene 1402 (e.g., comforting, Pavlovian cue to begin thinking about going to bed), wind down scene 1404 (e.g., where one swipe down initiates content optimized for winding down and falling asleep, and may include reduced lighting from the device), noise mask scene 1406 (e.g., automatically begins to help the user stay asleep, such as including an all dark setting), rescue 1408 (e.g., where a user is awake and not falling asleep, such as with racing thoughts, and where a rescue mode initiates a wind down sequence), sunrise alarm scene 1410 (e.g., presenting a wake light followed by a progressive alarm), alarm scene 1412, snooze 1414, and the like.

Scenes may be managed by the bedside sleep assistant and automated based on any combination of actions, states, events, and the like. For example, the bedside sleep assistant may provide management and automation based on any combination of:
A predefined sequence of scenes and IoT events called the “sleep routine” that is configured by default and optionally manually configured by the user.
Direct user interaction with the bedside sleep assistant (e.g., swiping down to begin the sleep routine).
The state of the user (presence, awake, asleep) as detected by sensory input embedded into the bedside sleep assistant, connected to the bedside sleep assistant, or wirelessly paired such as radar, acoustic, lidar, thermal, pressure sensors, accelerometers, and the like.
A pre-set schedule based on sleep data entered by the user.
The current state of the user based on information provided through third-party applications or functions (e.g., health APIs such as Apple's HealthKit, Google Fit, and the like).
Sensory input embedded into the bedside sleep assistant, connected to the bedside sleep assistant, or wirelessly paired such as radar, acoustic, lidar, thermal and/or pressure sensors, accelerometers, and the like.
Following guidance from a sleep restriction program input by a health professional.
Following guidance from a sleep restriction program input by the user.

Additionally in scenarios where the user is listening to content, such as an audio book, the bedside sleep assistant may pause playback if the user is detected as sleeping.

In embodiments, a ‘rescue’ scene 1408 may provide a sleep assistance when a user is having a difficult time falling asleep. People struggling with stress-induced insomnia or racing thoughts often have difficulty falling back asleep after a nighttime disruption. This can lead to prolonged wakefulness. Many people use their phones to play content to help them fall asleep, but this process can be counterproductive. Picking up the phone, unlocking it, navigating to an app, and selecting content can be stimulating, pulling the user into a more wakeful state. Additionally, checking email or social media can further increase sleep latency and consume valuable sleep time.

As part of automated scenes, the “Rescue” scene indicator may be displayed at the top of the user interface. If the user experiences a sleep disruption, they may start the Rescue scene with a single gesture. This scene plays content that the user has assigned or that the device automatically selects, aimed at distracting the user from their thoughts and helping them fall asleep more easily.

Once the Rescue scene has run its predefined duration or the device detects that the user has fallen asleep, the device may automatically resume the previous scene, such as noise masking, to help the user stay asleep. Alternatively, the user may manually swipe down on the scene indicator to return to the previous scene. The Rescue scene, once completed, may return to the previous scene, designed to assist users in falling back asleep after nighttime disruptions.

Another example of automatic behaviors is recommendations on when to nap. For instance, naps may be generally recommended if users experience daytime sleepiness, but there is an optimal time window to take a nap (generally in the early afternoon) and duration (about 20 minutes). The bedside sleep assistant may automatically present a nap indicator during the optimal time for a nap. For instance, take the last sleep data into account to offer the optimal time for sleep, automatically set a duration and use the wake up features to awaken the user, and the like. The bedside sleep assistant's behaviors may be initiated through a configurable schedule as well as direct interaction with the bedside sleep assistant. User preferences to guide behaviors may be set by sleep time, wake time, sleep duration, and the like.

### Automatic Behaviors and Internet of Things (IoT) Accessories

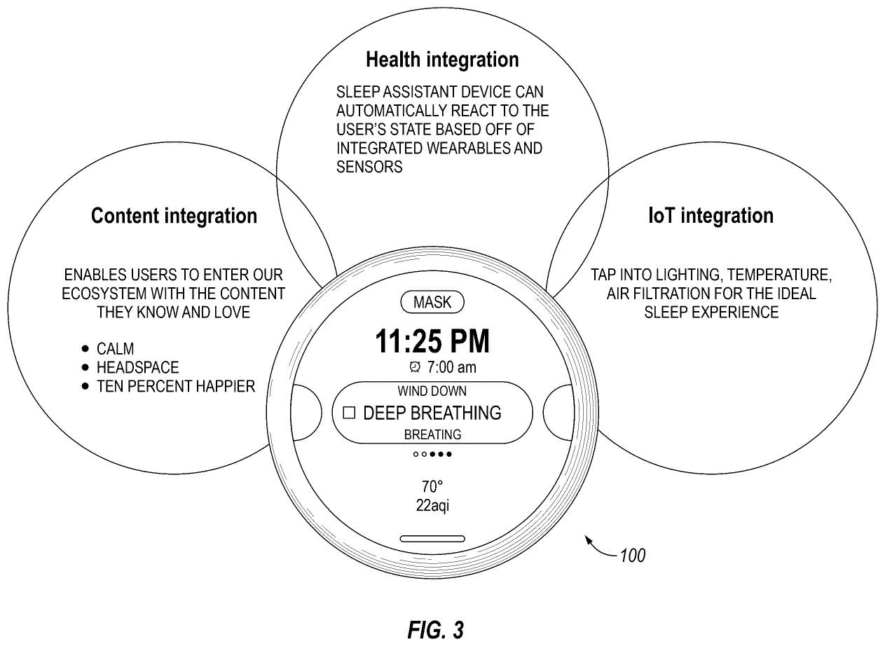

Referring to FIG. 3, the bedside sleep assistant 100 may provide an IoT connected hub by providing sleep support directly with the bedside sleep assistant and extending to IoT-connected devices.

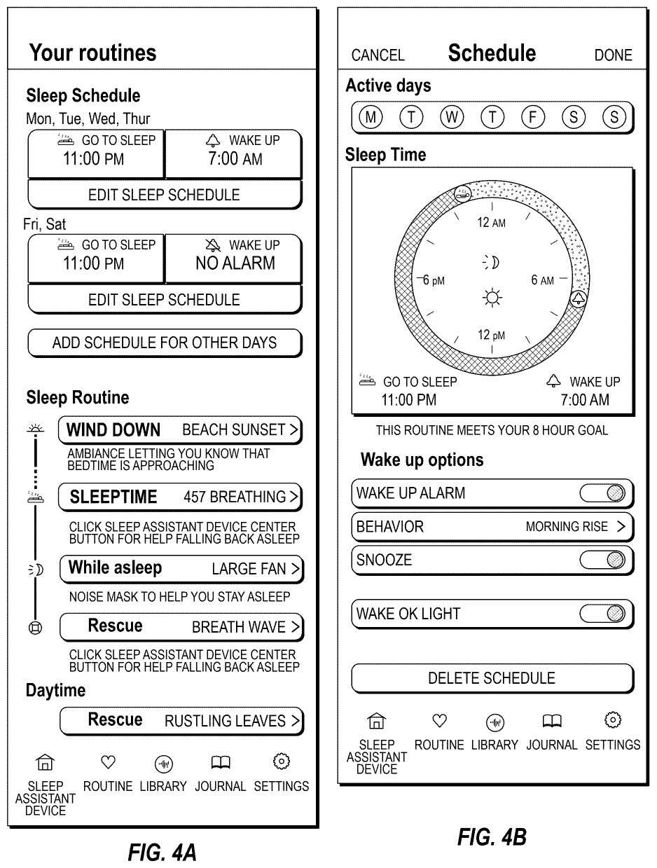

Referring to FIG. 4A-4B, the user may add IoT automation to the bedside sleep assistant's behaviors directly through the bedside sleep assistant, through an accompanying mobile interface, and the like. The bedside sleep assistant may communicate to paired IoT devices via IoT APIs and IoT protocols such as the Matter protocol through various networking protocols (WiFi, Bluetooth, Thread).

For example, when it's time for the user to sleep, the bedside sleep assistant may lower blinds, dim lights, adjust ambient temperature, and the like. When it's time to wake up, the bedside sleep assistant may trigger the blinds to rise and raise the ambient temperature.

### Multi-Device Interaction

A single bedside sleep assistant may operate standalone or in pairs for couples, or families, such as with one on each night table. A linked pair of bedside sleep assistants may enable shared functionality like lighting, stereo/spatial noise masking, individualized functionality like alarms, utility light, wind-down content, and the like. As such, the bedside sleep assistant may enhance the sleep experience and foster a supportive routine for both partners. Additionally, paired devices may share environmental and biometric data to improve accuracy of sensory data and the accuracy of biometric data such as but not limited to sleep data for each user.

Non-limiting example use cases include a couple that has two paired bedside sleep assistants, such as one on each night table, such as, for example:
When one partner starts a scene, both bedside sleep assistants will play back content in stereo, such as one stereo channel playing on one device and the other stereo channel playing on the other device.
In the morning, when an alarm begins, one bedside sleep assistant can sound the alarm, while the other partner's bedside sleep assistant continues to provide noise masking. Once the alarm is turned off, sound masking on both bedside sleep assistants may resume.
When the second partner wakes up and turns off the alarm, both bedside sleep assistants may stop noise masking.
When the alarm sounds, the second partner may remotely turn off the first partner's alarm. This is useful in the case the first user has gotten out of bed before their alarm sounds.
Each bedside assistant may have the same audio content but with different lighting experiences: For instance, one partner may use a wind down feature consisting of a lighting cue to help pace a breathing technique while both partners simultaneously listen to the same masking content.
One partner may listen to wind-down content via paired headphones, such as a spoken breathing exercise, while both the bedside sleep assistants are playing masking content in stereo, through their speakers. This avoids the need to use a mobile device to listen to sleep content via headphones.

The bedside sleep assistant may be paired with one or multiple other bedside sleep assistants and may be capable of linked and unlinked behaviors that cover audio, video, lighting, and the like. The bedside sleep assistant may be able to play back multiple tracks of audio simultaneously.

When bedside sleep assistants are paired, they may perform a combination of functions, such as:
Playing shared content in stereo, such as immersive ambient sounds for noise masking.
Layer additional content such as individual alarms, wind down routines, and the like.
Multiple bedside sleep assistants may react to each other based on what individual content is being played. For example, if the left partner's alarm is sounding, the right partner's bedside sleep assistant can adjust its sound masking to help the right partner stay asleep.
Adjustments like volume and lighting may be linked or unlinked between devices.
Bedside sleep assistants may be linked together for all or some lighting features. For instance, wake up lighting and alarms that are linked to both bedside sleep assistants.
Multiple bedside sleep assistants may monitor and react to environmental conditions based on information provided by sensors. Those sensors may be included in a bedside sleep assistant or can be separate IoT or other devices.
Sensor measurements from multiple devices may be combined to improve sleep data sensing precision.
Sensor measurements from multiple devices may be combined to add spatial information through triangulation, such as through a radar system in one or both devices.
Active sound masking: If one bedside sleep assistant detects offending noise via its microphone, paired bedside sleep assistants can actively adjust sound masking to reduce the perception of the offending noise.
Light exposure: If either bedside sleep assistant detects non-optimal lighting conditions for sleep, an action may be taken to, for instance, close shades, turn off lights automatically and the like.
With data from multiple devices, wearables, and sensors, and the like, the bedside sleep assistant may offer detailed feedback and personalized recommendations may be provided. This comprehensive insight can help users understand the various factors affecting their sleep and make informed adjustments to their routines. For example, if one partner experiences frequent sleep disruptions or snoring as detected by their device, the other user's device may highlight potential correlations in sleep challenges for both users, enabling them to address these issues more effectively.

In embodiments, radar-based system, utilizing radar systems in one or more bedside sleep assistants, may be used. For example, a radar-based may be provided, such as automatically activating a nightlight based on user presence and state detection. For instance, if one device detects that a user is present and in an “awake” state, it may automatically activate a nightlight across both devices. This feature helps ensure that the nightlight is only presented when needed and in a manner that minimizes disruption to a sleeping partner. The devices may be equipped with radar sensors that detect user presence and determine if the user is awake. When one device detects an awake user, it may trigger a nightlight on both devices. However, if one partner is sleeping, the device near the sleeping partner may remain inactive to avoid disturbing their sleep. The nightlight may only be activated on the device detecting the awake user if the other device senses that the partner is still sleeping. This setup helps ensure that the user has sufficient light to navigate the room safely without disturbing a sleeping partner. The radar-based detection provides a responsive solution that adapts to the users' needs and states, promoting better sleep hygiene and partner consideration.

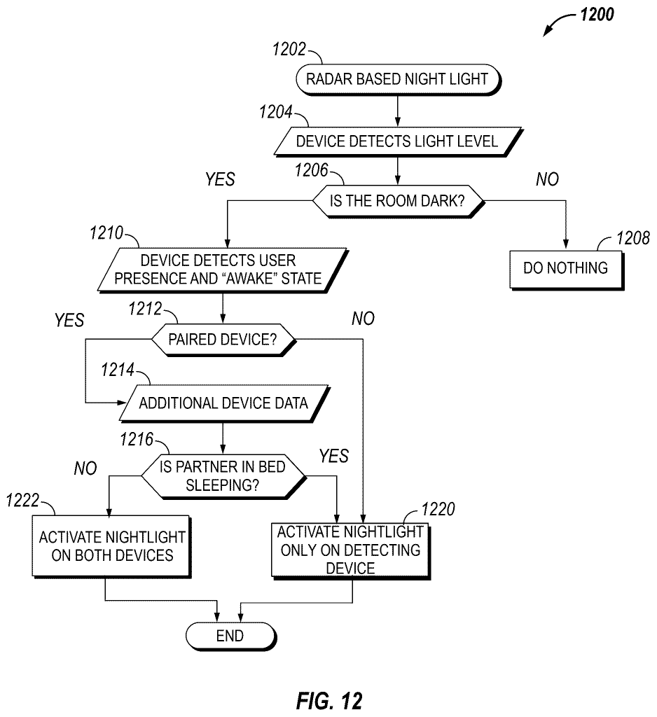

In an example embodiment, and referring to FIG. 12, a radar-based nightlight process sequence 1200 may include a start of the sequence 1202, where the device 100 detects a light level 1204 to determine if the room is dark 1206, and if the room is not dark, to do nothing 1208. If the device detects user presence and ‘awake’ state 1210 then the sequence continues to determine if the device is paired 1212, where if not, activates the nightlight on only detecting device 1220, and if yes, to continue to obtain additional device data 1214 to determine if the partner is bed is sleeping 1216, and if not sleeping, to activate the nightlight on both devices 1222, and if yes, sleeping, to activate the nightlight only on the detecting device 1220.

In embodiments, the bedside sleep assistant may utilize other sensors for either a single or linked configurations, such as temperature, radar, lidar, humidity, vibration, air quality, external sensors, and the like, to jointly monitor the user's environment and react to conditions to maintain proper sleep hygiene.

### Directed Light

The bedside sleep assistant's lighting system may improve the user experience of sleep, both for individual users and users sharing the room with a partner. Traditional bedroom lighting consists of a lamp that is either on or off, often projecting light at full brightness with light flooding the room. The bedside sleep assistant lighting system may enable precise control over brightness and/or the lighting direction, enabling the user to focus light where they need it so as not to interrupt the user's partner. When two bedside sleep assistants are used, such as by a couple sharing the same bedroom, the two sleep assistants may be interconnected through a communication system to coordinate the directionality and focus of lighting from the respective devices to shine light in the same manner (e.g., diffuse lighting directed to the ceiling of the room), to shine light to the same location (e.g., the lighting source for each device shining focused at a single place on a bed), and the like.

When connected with multiple external lighting systems, the directed light interface may connect with these systems to coordinate lighting effects and/or directionality across multiple lights, such as with recessed lighting, table lighting, and the like.

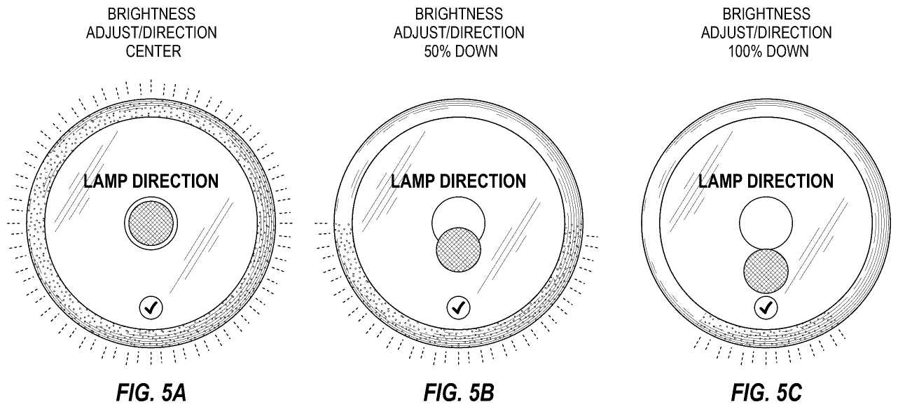

With respect to lighting control, and in a non-limiting example embodiment, and referring to FIGS. 1A-1D, the bedside sleep assistant's lighting system may consist of a capacitive touch strip, an interactive display, and an array of lighting elements:
Brightness touch strip: The user can turn on/off and adjust the brightness setting by running their finger up or down the touch strip.
Directionality: An onscreen adjustment is available to the user to position the light in any direction along the light ring. As an example, the user can direct the light down to illuminate only their night table so light does not project into the rest of the room. (see FIGS. 5A-5C)
Control ring: The control ring can also be used to change the direction of the light.
The position of the light is saved between interactions and restarting of the device.
The joystick can also be linked to other devices to either control multiple devices simultaneously or individually.
The directed light feature can be locally controlled on a device's display, wirelessly via mobile device, or physical control such as an integrated physical control such as but not limited to a capacitive input, physical encoder, joystick, dedicated remote, wall mounted light switch, and the like.
directed light feature can be incorporated into the sleep assistant, or in other IoT connected or standalone lighting products such as light bulbs, table lamps, floor lamps, light fixtures, LED light strips, automotive applications such as cabin interior lighting features or external lighting features, and the like.

The directed light on the sleep assistant may be controlled through the touchscreen display (see FIGS. 5A-5C). A touchscreen control for the directed light feature may be comprised of a virtual or physical “joystick” that can be positioned along x&y axis and an integrated brightness control slider. Additionally, brightness can also be adjusted with physical controls on the device such as a physical rotary control (control ring) or capacitive touch strip. By default, the joystick is positioned at the center of the display which illuminates all LEDs evenly. As the joystick is moved away from the center, the LEDs become more focused towards its position As the user moves the joystick in any direction, the light will concentrate in that direction (e.g., increasing the light on the control ring with integrated LEDs around the periphery of the control ring). Tapping the center of the display may return the joystick to the default position. The joystick may have an outer limit in which it can move so as not to block other UI elements.

The lighting system may automatically respond to the user's presence, position, and wake state. The lighting feature may automatically be turned, such as with an adjustable timer that can optionally work in conjunction with biometric sensing, for example when the user falls asleep or leaves the room. One or more devices may use radar and/or a combination of environmental sensors to detect the presence, distance, and/or position of one or more users. When a user enters the room, the device may sense presence, distance, position, and the like, relative to the device. Using this sensor information, the device may provide light for the user (i.e. in a dark setting) by adjusting brightness, color, spectrum, direction of light, and the like. One or more devices may use radar and/or a combination of environmental sensors to detect the state of the user, such as dimming the light when they fall asleep or providing a night light feature if the user awakens in darkness. Using this sensor information, the device may provide light for the user (i.e. in a dark setting) by adjusting brightness, color, direction, and the like, of light.

In an example of how a user may benefit from use of the directed light, a user may be preparing medication late at night while a partner is in bed. The user raises the brightness of the light using the device's capacitive strip, adjusting it precisely to the needed level. They also use the on-screen joystick to direct the light to a specific area, focusing it on the night table where the medication is placed. By setting the light to the necessary brightness and directing it accurately, the user can see clearly without disturbing their partner's sleep. After taking their medication, the user turns off the light by swiping down on the capacitive strip to fully dim it.

In an example, a user may be getting up in the middle of the night to use the restroom. The user experiences a mid-sleep awakening and needs to use the restroom. In a dark room with their partner sleeping beside them, the user reaches over to the device, feels the capacitive strip, and swipes up to gradually increase the lighting. To avoid shining light directly on their partner, the user activates the directed light feature, projecting light behind the device to cast a gentle, diffuse illumination across the room. This setup allows the user to navigate the bedroom safely to use the restroom and return to bed without disrupting their partner's sleep.

In an example, a user may be adjusting light for reading for both partners. One partner, already in bed, wants to set both devices to provide suitable light for reading for themselves and their partner. The user swipes up on the device's capacitive strip to reveal the directed light feature on-screen. They then press an on-screen button to control both paired devices simultaneously, adjusting the light direction and brightness level for optimal reading conditions. This setup ensures that both partners have the perfect lighting for reading without needing to adjust the lights individually.

In an example, a user may be setting up IoT directed light for watching a movie in the bedroom. Action: The user wants to watch a movie in the bedroom. Their ceiling has IoT controllable recessed lights. The user taps the capacitive strip on their bedside device to bring up the directed light feature on-screen. They then select the IoT lighting group for their bedroom. Using the on-screen joystick, the user dims the front lights and maintains a low, diffused light towards the back of the room. Outcome: This setup creates an optimal lighting environment for watching a movie, ensuring the front area is dimmed for better screen visibility while providing a gentle, diffused light at the back to avoid complete dark

### Internet of Things (IoT) & Application Integration

The bedside sleep assistant may provide the capability to interface with a multitude of IoT-based products, such as through Bluetooth connectivity, WiFi connectivity, and the like. This bedside sleep assistant may encompass support for common IoT networks and data protocols, offering the potential for integrations (e.g., integrated within the system, third-party integrations, and the like) to enhance the device's functionality. Such integrations may extend the bedside sleep assistant's command over third-party products, spanning a range of IoT networks and data protocols, applications, data storage and management systems, and user interfaces.

The bedside sleep assistant may provide the ability to integrate with third-party products through a menu-based system, such as where potential integrations are presented. For instance, a user may select the desired integration, after which the user gains access to the respective product's features through a bedside sleep assistant's interface. These features may also be appended to a bedside sleep assistant's suite of behaviors.

In a non-limiting example, a user might integrate a smart mattress with the bedside sleep assistant, programming the mattress to adjust temperature in synchrony with the onset of a sleep routine. In embodiments, adjustments to the mattress may also be made directly through the bedside sleep assistant interface, negating the need to interact with distracting mobile devices. For instance, as described herein, a sensed condition may trigger a scene change that runs through a routine of multiple actions including adjusting the temperature of the mattress, such as part of a rescue routine to help a user get back to sleep.

IoT and App integration provides benefits through its unique behaviors, software, and hardware architecture. These attributes confer upon users the capability to integrate products associated with mobile technologies, as well as the capacity to interact with various IoT connected devices and services, thereby creating the benefit of a user-centric and technologically advanced solution for sleep assistance.

### IoT Lighting Control Interface

The bedside sleep assistant's lighting interactions may extend to IoT-connected lighting, such as through an interaction to easily control the user's IoT lighting. This may be configured similarly to how the bedside sleep assistant's onboard lighting system works. The bedside sleep assistant may integrate IoT controls to extend convenience and ease of use to IoT lighting, such as the lighting in the user's bedroom.

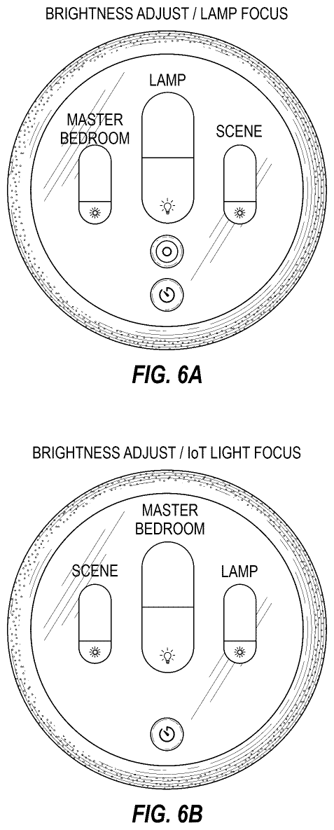

Referring to FIGS. 6A-6B, in a non-limiting example, when the user taps or swipes the brightness control strip, lighting controls appear on screen. Tapping the control strip may then toggle through lighting, for example, toggling through the bedside sleep assistant's utility light, behavior light, IoT connected lights, and the like. Tapping a minimized brightness bar may bring it to focus by scrolling the user interface (UI) element to the center of the display. Once the light is selected, the user may drag up or down to adjust brightness. Double tapping on the control strip may toggle on or off the selected light.

### Sleep Restrictions

Cognitive Behavioral Therapy for Insomnia (CBT-I) is a structured, evidence-based treatment designed to help individuals with insomnia identify and address the underlying causes of their sleep difficulties. Primary interventions in CBT-I typically involve a combination of cognitive and behavioral techniques.

Typically, recipients of sleep treatment through CBT-I and sleep hygiene recommendations are left to implement these treatments independently. The bedside sleep assistant may enable patients and healthcare professionals to enter CBT-I treatments into a platform for the bedside sleep assistant to act as an agent to help support sleep treatment. For instance, the solution may consist of the patient's bedside sleep assistant, a cloud-based service to host data and run web-based applications, a web interface for healthcare professionals, and the like.

Healthcare professionals may interact with the patient's account and submit parameters to program how the bedside sleep assistant will support the prescribed sleep interventions. The bedside sleep assistant may adjust its behavior to accommodate the prescribed sleep interventions. The bedside sleep assistant may automatically adjust its behavior to adhere to the prescribed sleep intervention. For example, if the patient is following a sleep restriction schedule, the bedside sleep assistant may automatically adjust sleep and wake times based on the user's performance based on information gathered by the bedside sleep assistant and/or through health API integrations and connected sensors. Healthcare professionals may optionally be granted the patient's data for review; modify sleep schedules, content, and training programs; and the like.

Automated sleep restriction may involve instructing the user on the optimal time to go to bed, limiting the time spent in bed to the actual time spent sleeping, and the like, thereby increasing sleep efficiency. As the user progresses through the sleep restriction treatment, adjustments may be made to optimize the user's sleep efficiency.

The bedside sleep assistant may act as an agent to help users, such as to adhere to CBT-I treatment and sleep hygiene. When coupled with the sleep assistant's automated behaviors, the bedside sleep assistant may help users improve their sleep quality by optimizing the time spent in bed. By following a structured process, the bedside sleep assistant may collect essential information about the user's sleep habits and patterns and then tailor recommendations to enhance sleep efficiency. Gradually, the bedside sleep assistant may adjust its behavior to help increase the time spent in bed until a healthy sleep duration is achieved.

Sleep compression is a behavioral treatment designed to improve sleep efficiency and promote contiguous sleep by initially reducing the time spent in bed. This method helps individuals who have trouble falling asleep or staying asleep by concentrating their sleep period and gradually expanding it as their sleep quality improves.

The bedside sleep assistant may monitor the user's sleep patterns to guide them through a sleep restriction program. These sleep patterns may include:
Sleep Latency: The amount of time it takes to fall asleep.
Bedtime and Wake-up Time Variability: The consistency of sleep and wake times.
Sleep Contiguity: Periods of sleep disruption throughout the night.
Total Amount of Sleep: The total hours of sleep per day.
Sleep Debt: The accumulation of missed sleep over a period of time, typically a span of 2 weeks.

The bedside sleep assistant may track these sleep patterns to support users in adhering to a sleep restriction program, ultimately helping them achieve their sleep goals, where the bedside sleep assistant may provide feedback and recommendations, enabling users to adjust their sleep routines and improve their overall sleep quality.

The sleep restriction program may be adapted to support other common temporal sleep challenges such as the user deviating from their normal sleep schedule, and jet lag.

Radar may be used to monitor the user's movements and breathing patterns without physical contact. This allows the bedside sleep assistant to detect, for instance, when the user falls asleep and wakes up, providing sleep data. Radar may also detect restlessness or disturbances during the night, helping to identify patterns that may be affecting sleep contiguity. By combining this data with sleep restriction protocols, the bedside sleep assistant may tailor recommendations and adjustments to the user's sleep environment and routine to help them meet their sleep goals.

With data from multiple devices, wearables, and sensors, the bedside sleep assistant may offer detailed feedback and personalized recommendations. This comprehensive insight can help users understand the various factors affecting their sleep and make informed adjustments to their routines. For example, if one partner experiences frequent sleep disruptions or snoring as detected by their device, the other user's device may highlight potential correlations in sleep challenges for both users, enabling them to address these issues more effectively.

The bedside sleep assistant may guide users in setting sleep restriction goals, such as target bedtime, wake-up time, total sleep time, and the like. The user may track their progress during the intervention, adjusting as necessary based on the bedside sleep assistant's feedback. The bedside sleep assistant may then support users in implementing these goals through bedtime reminders, wake-up alarms, progress tracking, and the like.

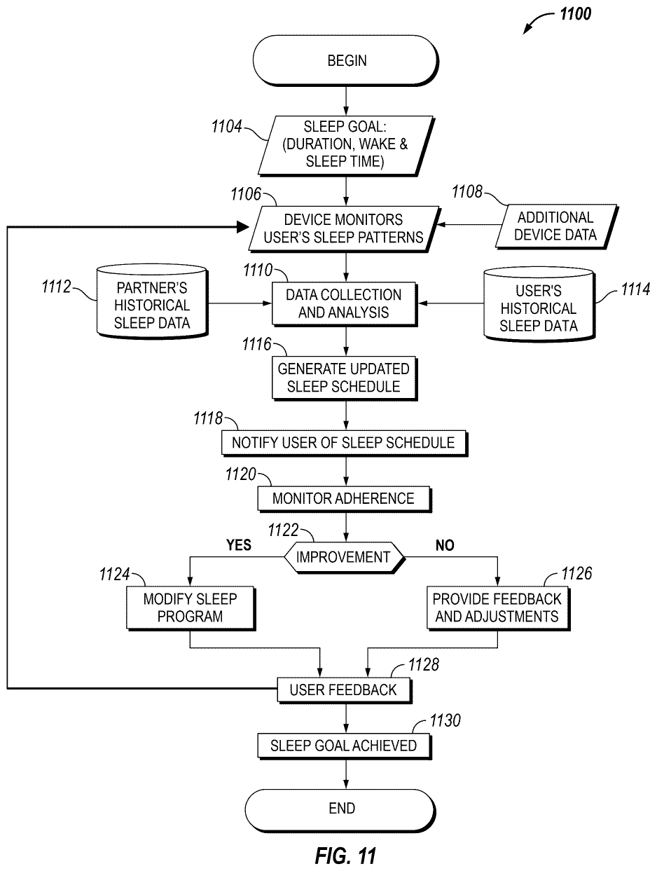

Referring to FIG. 11, an embodiment sleep restriction sequence 1100 is depicted, where the beginning of the sleep restriction program determines a sleep goal 1104 (e.g., duration, wake, and sleep time). The device 100 then monitors the user's sleep patterns 1106. In embodiments, additional data 1108 may be provided for the monitoring. The user's historical sleep data 1114 and the partner's historical sleep data 1112 are incorporated to perform data collection and analysis 1110. An updated sleep schedule may be generated 1116, where the user is notified of the sleep schedule 1118.

Adherence is then monitored 1120 to determine improvement 1122. If no improvement is found, then feedback and adjustments are provided 1126. If improvement is found, then the program is modified 1124. User feedback 1128 may be provided back to step 1106 1120. If the sleep goal is achieved 1130, then the sleep compression program is ended.

The bedside sleep assistant may provide progress tracking, such as where the bedside sleep assistant tracks users' progress during the sleep restriction intervention. Users may review their sleep data and efficiency on the display to monitor improvements and adjust as necessary. The bedside sleep assistant may provide feedback and encouragement to keep users motivated throughout the process.

The bedside sleep assistant may provide customization by offering various customization options for users, such as selecting preferred alarm tones, adjusting night light settings, tailoring sleep restriction goals to individual needs, and the like.

### Journaling and Automated Recommendations & Behaviors

The bedside sleep assistant may periodically prompt the user to log and assess the quality of their sleep, such as through one or more questions. The user may input text in the form of general voice and/or text entries, reminders, and to-do lists to help reduce stress and enable the user to fall asleep more easily. This information may be time-stamped and stored on the bedside sleep assistant and/or in the cloud and retrieved on the bedside sleep assistant or its accompanying mobile application.

Journal entries may also capture the context around the user's environment, state of the bedside sleep assistant, time & date, and the like. Entries may enable the user to easily recount the information they logged via the bedside sleep assistant or associated mobile application and receive recommendations from the bedside sleep assistant.

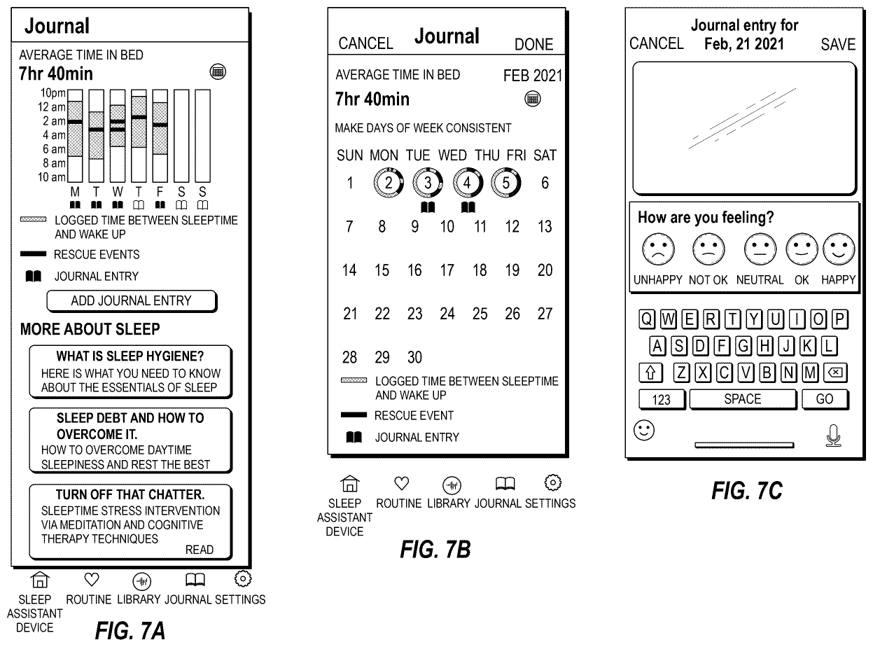

The bedside sleep assistant or bedside sleep assistant's mobile application may prompt the user for a journal entry after they have awoken. A simple restfulness score may be recorded as part of this daytime journal entry. Referring to FIGS. 7A-7C, this information may help the bedside sleep assistant recommend successful sleep interventions or downrank interventions that do not generate neutral or positive results for the user. The bedside sleep assistant may take this information and summarize the user's sleep habits in the mobile app or on-screen on the bedside sleep assistant.

Additionally, recommendations based on the user's sleep metrics and journaling entries may be used to monitor sleep health as well as make recommendations. These responses may be generated locally on the bedside sleep assistant such as through text or voice feedback, for example, as “what seems to be working”, “what could use improvement”, and the like.

Journal entries may be entered through the bedside sleep assistant's onboard journaling app or through an accompanying mobile application. The journaling app on the bedside sleep assistant may allow for input via an onscreen keyboard. Voice interactions may be recorded as an audio recording or via text to speech.

Additional information may be recorded in the journal entry, such as the user's sleep goals, sleep metrics, environmental conditions, the state of the bedside sleep assistant, what content (if any) was being presented, the date and time, and the like.

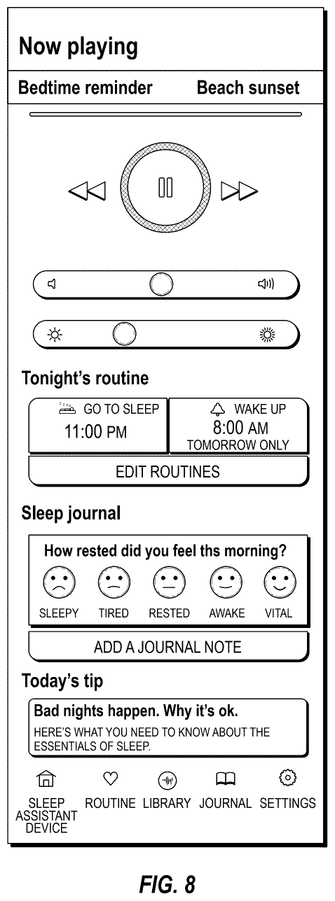

Once the user has awakened, the bedside sleep assistant may provide an onscreen shortcut to the journal entry for easy retrieval. Shortcuts may improve the efficacy of journaling and make to-do list items more actionable. Referring to FIG. 8, the user's journal entries may be shared through various means, such as email, SMS, an accompanying mobile application, and the like.

Automated recommendations and behaviors may be made to the user based on sleep metrics gathered by a plurality of sensors, APIs and journaling. A summary of the previous night may be recalled through the bedside sleep assistant or mobile app. An automated summary may be generated using large language model (LLM) neural networks. The bedside sleep assistant may react to the user's journal by providing content, and feedback as the user progresses through sleep goals as well as areas where they may be able to make improvements.

### Dynamic Content

Breathing techniques are highly effective at deactivating the sympathetic nervous system, which enables shorter sleep onset latency (time to fall asleep). The general recommendation is a breathing technique of six breaths per minute. However, this breathing pace may be too fast or too slow for the user.

The bedside sleep assistant may utilize audio and/or video content and lighting cues to help guide the user's breathing pace. For example, the user may adjust the pacing of content consisting of wave sounds and accompanying lighting without affecting sound quality.

For narrated content, speech pace, and overall spacing of words, sentences, and paragraphs, may be dynamically adjusted based on the user's preference for total duration of the content.

Sounds on the bedside sleep assistant may consist of samples and synthesized sound that can be programmatically played, similar to sound reproduced by musical instruments known as synthesizers and samplers. When adjustments are made, the periodicity of sounds may slow or speed up without altering the pitch or degrading sound quality. The lighting system may be time-aligned with the shifted audio content. The programmatic elements may be overlaid on additional soundtracks such as environmental recordings.

In embodiments, detection of the users breathing rate and using that rate may be used as the starting point. For instance, The breathing rate may try to guide the user to no less than 6 breaths per minute. Additionally, the user may prefer to manually set the starting breathing rate, and target breathing rate, and transition time to meet that breathing target. The device may automatically suggest optimal start and target breathing rates based on historical data pulled from data from other API integrated health products, sensors, wearables, or from the devices own sensor data including radar data. Additionally, breathing rate goals may be set that the user can work towards over time as part of a breathing improvement program. Additionally, spoken content may be compressed or expanded based on the user's wake state and/or wind down duration preference. This content can be adjusted offline or in realtime using a large language model, text to speech algorithm, pre-recorded, looping audio, generative audio, and the like, such as mixed together to fill the appropriate time and/or state of the user.

### Sound Journey

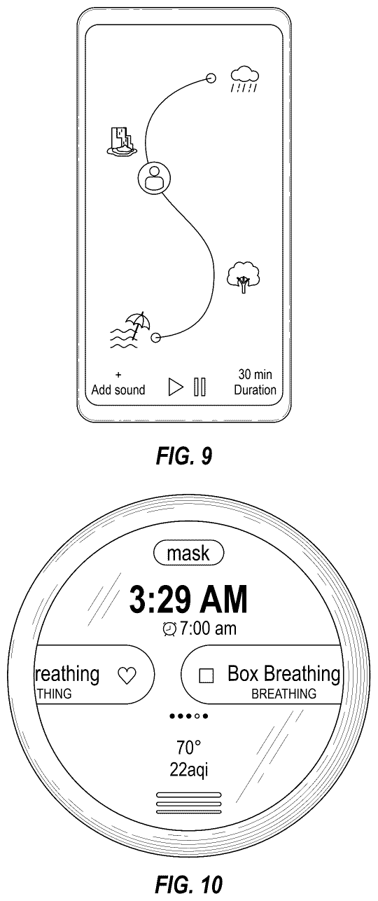

A common method for falling asleep is mental imagery, imagining a calming scene or journey to help calm the mind. The bedside sleep assistant and accompanying app may allow users to create a custom sonic soundscape they can experience as a sleep aid. Additionally, and referring to FIG. 9, soundscapes may be used for various environmental backgrounds to aid with studying, working, relaxing, and the like.

The user may create the sound journey by using the accompanying mobile app or directly on the bedside sleep assistant. The user may drag and drop sounds and/or lighting elements into the journey, placing them in succession or overlaid to create environmental soundscapes. The user may draw a path between sounds that creates the path through the sound journey. As the journey progresses, the proximity of the listener to the sound may increase and decrease in volume based on distance. Sound journeys may be played back in stereo, through spatial sound, or surround sound reproduction depending on what audio reproduction bedside sleep assistant is being used. The sound journey may be automatically modified by the bedside sleep assistant depending on the user's sleep state by adjusting pace or sound type. The sound journey may be started, stopped, or paused based on the user's detected state. Sound journeys may be played back on the bedside sleep assistant or accompanying mobile application.

### Single Interaction Content Selection

When the user changes content on the bedside sleep assistant, current and next (or previous) tracks may be blended (mixed), such as depending on the state of a swipe transition. For example, when the user swipes only half to the next track, the current and the next track are mixed with equal power. Similarly, LED light animations may be “pushed” left and right during track changes and up and down during scene changes.

Flipping through content is usually done by swiping or clicking a “next” or “previous” button: the previous audio track stops, and the next starts. Referring to FIG. 10, changing the volume and mixing different tracks dynamically through swipe transitions may let the user preview (“peek”) at adjacent content and presents a new, fun way to interact with a track list.

### Scene Interactions

The bedside sleep assistant may provide scene interactions through use of a microphone located at the top of the bedside sleep assistant (see FIGS. 1A-1D). If the feature is enabled, the microphone can listen for the sonic characteristics of a blowing sound, similar to white noise. Long periods of detected white noise may trigger an interaction, such as a disturbed candle flame. Short bursts of white noise may execute another action, such as “blowing out” (e.g., stopping) the content.

In this way, scenes may be turned off or “blown out” when the user blows gently on the top of the bedside sleep assistant. For instance, a long blow can disturb the lighting content and trigger sound effects, similar to partially blowing out a flame. A short blow, perceived as a burst, may “blow out” the content, stopping playback, light, and/or starting different content. For example, blowing on the top of the bedside sleep assistant will turn off a simulated candle or fire, or a long blow will ‘disturb’ a flame.

### Sleep Apnea Detection & Monitoring

Obstructive sleep apnea (OSA) affects over 39 million people in the U.S., however it can be largely undetected. Moreover, with lifestyle changes, age, weight, and overall health, OSA can be intermittent and challenging to detect. Despite adequate access to health care, 80% of moderate to severe OSA cases remain undiagnosed. OSA is linked to several adverse health conditions, such as hypertension, stroke, congestive heart failure, coronary artery disease, cardiovascular mortality, insulin resistance, and neurocognitive dysfunction.

Today's medical solutions consist of specialized medical equipment for use in labs, in-home diagnostic equipment. There are also consumer offerings from mobile apps to wearables that offer various forms of OSA pre-screening.

Consumer products for detecting obstructive sleep apnea (OSA) face several challenges in accuracy, reliability, and diagnostic capability. These devices often have limited sensor capabilities, leading to false positives or negatives and inconsistent data due to environmental factors and user compliance issues. Additionally, some of these products consist of wearables which can lead to inconsistent use due to discomfort, charging, and user hesitation to wear devices in bed. Bedside solutions may have a limited user interface as well as require a mobile app for viewing detailed information. These pain points can lead to inconsistent measurement and adherence to such a monitoring system.

Moreover, when two people share a bed, false data can be generated due to the presence of a partner. Contactless solutions that use microphones and radar may incorrectly detect patterns because of the other person in the bed.

In embodiments, the bedside sleep assistant is a standalone device that integrates a variety of sensors to monitor and enhance the user's sleep experience. It features advanced sleep assistant functions capable of tracking the user's presence in bed, movement, sleep stages, breathing patterns, and snoring.

By combining radar sensor data with microphone data, the bedside sleep assistant may detect episodes of snoring by identifying and monitoring snoring sounds, monitor respiratory rate and track breathing patterns to detect lapses in consistent breathing and signs of respiratory effort, adjust volume for monitoring when playing content like noise masking, the device may automatically lower its volume and that of any paired devices to ensure the microphone can effectively listen for breathing and snoring sounds, and the like.

For users sharing a bed, the bedside sleep assistant may differentiate between the snoring of each partner, where, for instance, each device monitors the breathing patterns using radar and matches them to the detected snoring sounds to determine the source. In an example, partner 1's sleep assistant detects snoring and matches the sound pattern to radar data indicating chest movement. Partner 2's sleep assistant also picks up the snoring sound but detects a mismatch in breathing pattern and frequency, allowing it to reject the data as not originating from Partner 2. This dual-device setup ensures accurate monitoring and helps prevent false detections, providing a solution for managing and understanding sleep health.

### Personalized Artificial Intelligence (AI) Generated Sleep Story Creation and Delivery

Artificial intelligence (AI) based content generation may be provided, such as, for example, for the creation and delivery of personalized sleep stories designed to aid sleep and relaxation, where a sleep story is a kind of narrative that is designed to help individuals fall asleep. It may involve soothing voice narration, descriptive language, slow-paced storytelling, and the like, that aims to take a user's mind away from the stresses and anxieties of the day. A goal of the story is to shift the focus from anxieties or preoccupations, creating a peaceful state of mind that encourages sleep. The content of sleep stories may vary greatly, with some sleep stories being pure fiction, and others involving guided imagery, mindfulness practices, current events, or even educational material presented in a calming manner.

In embodiments, customized sleep stories may be created using a plurality of AI generative tools. This content may be delivered to hardware platforms such as the bedside sleep assistant, associated software platforms, and the like. This may utilize AI generative tools to create and deliver customized sleep stories. User inputs may guide the content creation, allowing for personalized narratives based on factors like the age of the listener, preferred language, duration of the story, the option for automated series generation based on the user prompt, biofeedback from the user using a plurality of sensors to detect wake state, and the like.

In an example use case, the user may provide inputs. For example, users can create a customized sleep story by providing a prompt through text entry or voice input. This input can occur on a designated sleep assistant device, through an accompanying app, or passed through an API integrated with other assistant platforms such as Siri, Google Assistant, and Alexa Voice Services.

The user may provide a prompt input. For example, the user can create a customized sleep story by inputting a prompt via text entry or voice input either on the bedside sleep assistant or through an accompanying app, or passed through an API integrated with other assistant platforms such as Siri, Google assistant, and Alexa voice services.

A wrapper prompt may be provided. For example, a “wrapper prompt” or “system message” serves as a meta-instruction guiding the AI model's behavior, setting the tone, style, or specific guidelines for the user-prompt output. This prompt instructs the AI to generate a script and associated media assets including narrative text, voice narration, album artwork, imagery or video clips, LED lighting content for the sleep assistant, and timecodes and duration for asset placement.

Content may then be packaged. For example, the generated assets are delivered to AI tools for generation. Once created, the system references the timecodes for the assets and constructs a content package for the AI-generated sleep story. These prompts are delivered to AI generation tools for asset generation. Once assets are created, the solution references the timecode for the assets and constructs a content package for the AI generated sleep story.

The stories may then be delivered. For example, upon creation, these sleep stories are stored in the user's content library. Users can access, share, and play this content from any device connected to the bedside sleep assistant or logged in with the user's credentials. AI-generated content can also be shared via social media or similar platforms.

Personalized AI generated sleep story creation and delivery provides an improved means of generating and delivering personalized sleep stories to aid sleep and relaxation and represents an advancement in the application of artificial intelligence for personalized content creation.

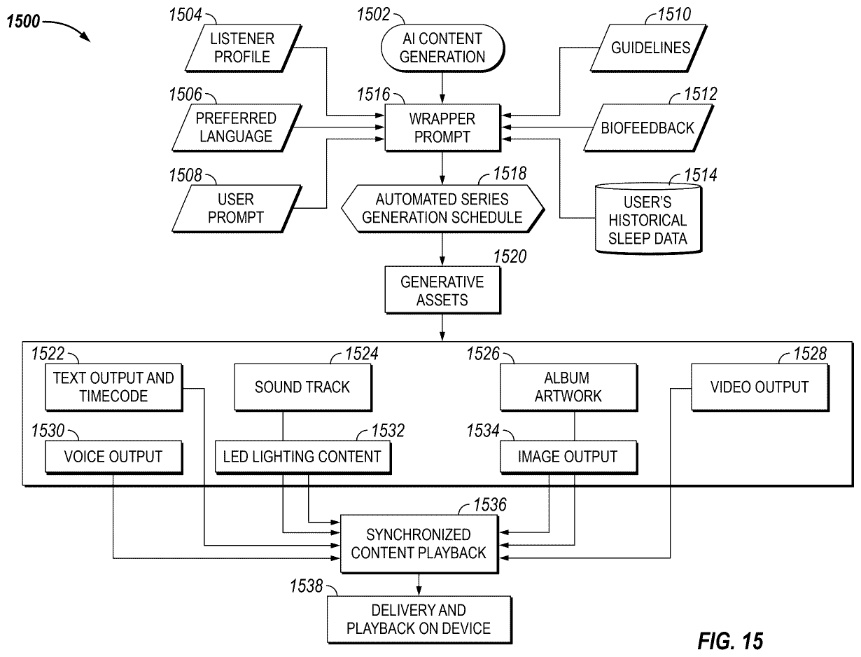

In embodiments, and referring to FIG. 15, an example AI generated sleep story generation and delivery is depicted 1500. AI content generation 1502 may begin with a wrapper prompt 1516, such as including inputs such as a listener profile 1504, preferred language 1506, a user prompt 1508, guidelines 1510, biofeedback 1512, a user's historical sleep data 1514, and the like. An automated series schedule 1518 and assets 1520 may be generated. Synchronized content playback 1536 may be produced from a plurality of sources, such as text output and timecode 1522, sound track 1524, album artwork 1526, video output 1528, voice output 1530, LED lighting content 1532, image output 1534, and the like. Delivery and playback 1538 to the device 100 may then be initiated.

### Non-Limiting Embodiments

In embodiments, the bedside sleep assistant device may include a plurality of components described herein, e.g., a user interface (e.g., control ring, touchscreen display, touch strip, communication system to external devices or another bedside sleep assistant device) a lighting system (e.g., LED ring), audio system (e.g., microphone, speaker), monitoring sensor system (e.g., radar, humidity, temperature, ambient light), a housing; and the like. The bedside sleep assistant device may include a controller (e.g., a computer, computing device, processor, circuit, and the like), such as including a processor and a memory, the controller configured to store a set of instructions that, when executed, cause the controller to execute, for instance, one or more sleep assistance functions, as described herein. The bedside sleep assistant device may include a communications system configured to exchange data with external devices, such as a second bedside sleep assistant device, an external device connected to a network (through wired or wireless connection), to an Internet of Things (IoT) device (e.g., lighting device, dimming device, blinds, media systems, sound systems, and the like).

### Bedside Sleep Assistant Device

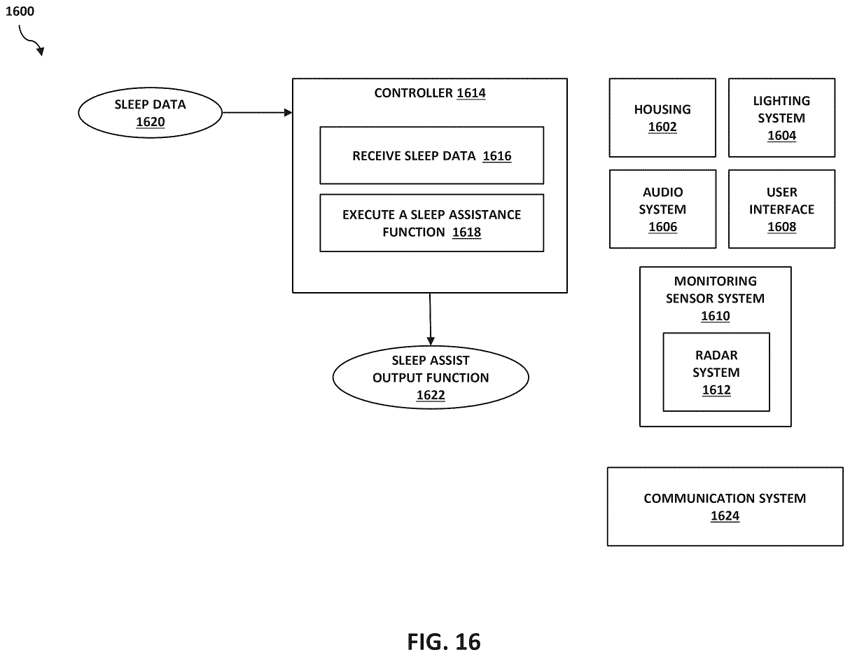

In embodiments, and referring to FIG. 16, the techniques described herein relate to a bedside sleep assistant device 1600, including: a housing 1602; a lighting system 1604 integrated with the housing; an audio system 1606 integrated with the housing; a user interface 1608 for receiving user commands; a monitoring sensor system 1610 for monitoring a sleep state, wherein the monitoring sensor system 1610 has an operating area including a user sleep area; and a controller 1614 including a processor and a memory, the controller configured to store a set of instructions that, when executed, may cause the controller to receive 1616 sleep data 1620 from the monitoring sensor system 1610, and execute 1618 a sleep assistance function that performs a sleep assist output function 1622 based on the received sleep data 1620. The sleep assistance function and/or sleep output function may be based at least in part on one or more method or system for sleep assistance as described herein.

In some aspects, the techniques described herein relate to the bedside sleep assistant device 1600, wherein the sleep data 1620 may include user presence data for at least one user in the user sleep area. The user presence data may be a user motion. The user presence data may be a user micro motion. The user micro motion may be one of a breathing or heartbeat user motion. The sleep data 1620 may further include sleep environmental data.

In some aspects, the techniques described herein relate to the bedside sleep assistant device 1600, wherein the sleep data 1620 may further include sleep preference data as provided by a user.

In some aspects, the techniques described herein relate to the bedside sleep assistant device 1600, wherein the user interface 1608 may include at least one of a tactile control input and a touchscreen display. The tactile control input may be a control ring, the control ring and the touchscreen display mounted on the housing, where the touchscreen display may be mounted to a front surface of the housing and the control ring mounted proximate to and surrounding the touchscreen display, where a tactile command input received through at least one of the control ring and the touchscreen display may at least in part determine the execution of the sleep assistance function. The received tactile command input may be a swiping motion on the surface of the touchscreen display. The received tactile command input may be a tapping motion on the surface of the touchscreen display. In some aspects, the bedside sleep assistant device 1600 may further include a control touch strip on the surface of the housing, where the received tactile command input may be a sliding motion on the surface of the control touch strip.

In some aspects, the techniques described herein relate to the bedside sleep assistant device 1600, wherein the monitoring sensor system 1610 may be further configured to sense a gestural movement of a hand in proximity to the housing. A sensed gestural movement of the hand may be received as a contactless command to wake up the bedside sleep assistant device. A sensed gestural movement of the hand may be received as a contactless command to change a brightness level of the display. A gestural movement may be a swiping motion of the hand proximate to the bedside sleep assistant device. A camera system may be used for sensing a gestural movement. A sleep routine may execute a pre-determined series of actions by the controller. The sleep routine may be a rescue routine, where the pre-determined series of actions by the controller presents the actions to a user to help the user get back to sleep. The gestural movement of the hand may trigger a command to an external device. The external device may be an internet of things (IoT) external device. The monitoring sensor system 1610 may include a radar system 1612 for monitoring the sleep state in the operation area including the user sleep area. The monitoring sensor system 1610 may sense a user's movements, where the user's movements may at least in part determine the execution of the sleep assistance function. The monitoring sensor system 1610 may sense a user's breathing patterns, where the user's breathing patterns may at least in part determine the execution of the sleep assistance function. The monitoring sensor system 1610 may sense when a user falls asleep, where the sensing of the user falling asleep at least in part determines the execution of the sleep assistance function. The sleep assistance may function as a change in at least one of a lighting parameter of the lighting system 1604 or an audio parameter of the audio system. The monitoring sensor system 1610 may sense a user sleeping restlessly, where the sensing of a user sleeping restlessly may at least in part determine the execution of the sleep assistance function. The sleep assistance function may initiate a sleep routine that executes a pre-determined series of actions by the controller. The sleep routine may be a rescue routine, where the pre-determined series of actions by the controller presents the actions to a user to help the user get back to sleep. The monitoring sensor system 1610 may sense when a user is awake, where the sensing of the user being awake may at least in part determine the execution of the sleep assistance function. The sleep assistance function may trigger a command to an external device. The external device may be an internet of things (IoT) external device. The sleep assistance function may be a change in a lighting effect of the lighting system. The change in the lighting effect may be changing an illumination direction of the lighting system. The change in the lighting effect may be changing an illumination brightness of the light system. The sleep assistance function may be a change in an audio parameter. The audio parameter may be a change in an audio volume.

In some aspects, the techniques described herein relate to the bedside sleep assistant device 1600, may further include a second bedside sleep assistant device including a second user interface, a second monitoring sensor system, and a second controller, where the bedside sleep assistant device and the second bedside sleep assistant device each further include a communication system 1624 configured to exchange data between the bedside sleep assistant device and the second bedside sleep assistant device, wherein a second sleep data 1620 may be received from the second bedside sleep assistant device through the communication system of the bedside sleep assistant device, wherein the execution of the sleep assistance function may in part be determined by the received second sleep data. A sensed condition by the second monitoring sensor system 1610 may be communicated to the bedside sleep assistant device, where the sensed condition may at least in part determine the execution of the sleep assistance function in the bedside sleep assistant device. The sleep assistance function in the bedside sleep assistant device may be coordinated with a second sleep assistance function in the second bedside sleep assistant device. The sleep assistance function and the second sleep assistance function may be a lighting effect of the lighting system. The lighting effect may be a coordinated directional lighting between the lighting system 1604 and a second light source. The lighting system 1604 may be integrated into the housing of the bedside sleep assistant device and the second light source integrated into the housing of the second bedside sleep assistant device. The sleep assistance function and the second sleep assistance function may be an audio effect. The audio effect may be a stereo effect, with one stereo channel of a soundtrack playing on the bedside sleep assistant device and the other stereo channel of the soundtrack playing on the second bedside sleep assistant device. In some aspects, a second sleep assistance function in the second bedside sleep assistant device may be mirrored in the sleep assistance function in the bedside sleep assistant device. The second monitoring sensor system 1610 may sense a second operation area including a second user sleep area.

In some aspects, the techniques described herein relate to the bedside sleep assistant device 1600, further including a wireless communication system in communication with an external device, where an external command received from the external device may at least in part determine the execution of the sleep assistance function in the bedside sleep assistant device. The external device may be a mobile phone. A wireless communication system may be in communication with an external device, wherein the sleep assistance function may in part cause the controller to transmit a command to the external device through the wireless communication system. The external device may include an alarm feature, and the command to the external device disables an alarm. In some aspects, the controller may be further configured to execute a sleep routine as part of the sleep assistance function, wherein the sleep routine may include transmitting a command to the external device to change from a first configuration to a second configuration. The sleep routine may further include sending a second command to the external device to change from the second configuration to the first configuration when a monitored condition is received. The external device may be an internet of things (IoT) device for controlling a window covering state, the first configuration may be a window covering state that covers the window, the second configuration a window covering state that uncovers the window, and the monitored condition an alarm indication. The sleep routine may be initiated when an initiating condition is received, the sleep routine may transmit the command to the external device to change from the first configuration to the second configuration when a monitored condition is received, and the sleep routine may transmit a second command to the external device to change from the second configuration to a third configuration when a second monitored condition is received. The external device may be a media device configured to play a media, the initiating condition the monitoring sensor system 1610 sensing a user in the user sleep area has fallen asleep, the first configuration the media device playing the media, the second configuration the media device stopping the playing of the media, the second monitored condition the monitoring sensor system 1610 sensing a user in the user sleep area has awoken, and the third configuration performing a partial rewind of the media and then continuing to play the media. The external device may include a sleep routine, the command to the external device begins the sleep routine, and a second command to the external device disables an alarm. The sleep routine may be a rescue routine, wherein a pre-determined series of actions by the controller may present the actions to a user to help the user get back to sleep.

In some aspects, the techniques described herein relate to the bedside sleep assistant device 1600, wherein the monitoring sensor system 1610 may further include a radar system 1612, the second monitoring sensor system 1610 further including a second radar system, where the radar system 1612 detects a motion of a user in the user sleep area and the second radar system detects a motion of a second user in a second user sleep area, where the controller may be further configured to differentiate a sleeping pattern of the user from a second sleep pattern of the second user based on the received sleep data 1620 and received second sleep data, where the execution of the sleep assistance function is based at least in part on the differentiation. The monitoring sensor system 1610 may further include a microphone for receiving a sound pattern, where the controller is further configured to compare the received sound pattern to the motion detected by the user and the motion detected by the second user and determine if the sound pattern matches either the motion of the user or the motion of the second user, where the execution of the sleep assistance function is based at least in part on the determination of which user motion matches the received sound pattern. The sleep assistance function may be based at least in part on the determination that the motion of the user in the sleep area monitored by the bedside sleep assistant device is a match to the received sound pattern. The match may be an indication that the user is snoring and the second user is not snoring. The match may be determined based at least in part by a match of a frequency determined from the motion of the user and a frequency determined from the sound pattern. The controller may be further configured to initiate a sleep routine that executes a pre-determined series of actions by the controller. The sleep routine may be a rescue routine, where the pre-determined series of actions by the controller presents the actions to a user to help the user get back to sleep. The sleep assistance function may be based at least in part on the determination that the motion of the user in the sleep area monitored by the bedside sleep assistant device is determined to be a mismatch to the received sound pattern. The mismatch may be an indication that the second user is snoring and the user is not snoring.

In some aspects, the techniques described herein relate to the bedside sleep assistant device 1600, wherein the sleep assist output function may control a media playback function. The control function may be stopping the media playback when the bedside sleep assistant device determines a user in the user sleep area has fallen asleep. The bedside sleep assistant device may determine a user in the user sleep area has fallen asleep at least in part through a sleep state change detected by the monitoring sensor system. The playback may be partially rewound upon a re-start of the media playback. A command may be output to an external media device. An artificial intelligence (AI) generated sleep story may be created, where the controller may be further configured to play the AI generated sleep store through the audio system. The AI generated sleep story may be created utilizing a generative AI tool. Further including the user interface 1608 connected to a wireless network, where the generative AI tool may be accessed through the wireless network. The access may be through an application programming interface. The AI generated sleep story may be created at least in part from a user input specifying at least one of an age of a listener, a preferred language, a duration of the story, an option for the AI generative sleep story to be a series-based story, and the like. The AI generated sleep story may be executed through audio presentation through the audio system. The AI generated sleep story may be executed through audio presentation through the audio system 1606 and through a visual presentation through the lighting system.

### Directed Lighting

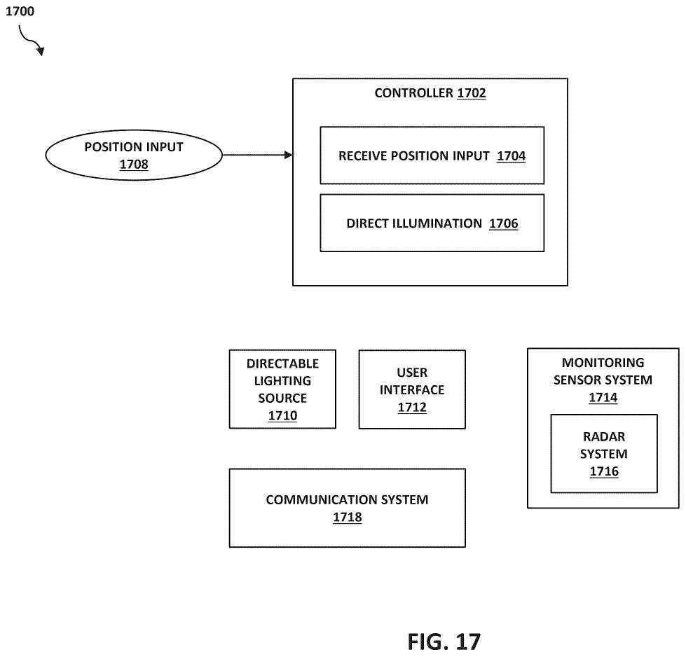

In embodiments, and referring to FIG. 17, the techniques described herein relate to a lighting system 1700, including: a user interface 1712 configured to receive a position input 1708; a directable lighting source 1710, wherein an illumination direction of the directable lighting source is electronically directable; and a controller 1702 including a processor and a memory, the controller 1702 configured to store a set of instructions that, when executed, may cause the controller to: receive 1704 the position input from the user interface; and direct 1706 the illumination direction of the directable lighting source 1710 to correspond to the received position input 1708 from the user interface 1712.

In some aspects, the techniques described herein relate to the lighting system 1700, wherein the directable lighting source 1710 may be a light emitting diode (LED) array. The user interface 1712 may include a touchscreen display, wherein the directed illumination direction of the directable lighting source 1710 corresponds to the received position input 1708 received through the touchscreen display. The lighting system may further include a housing, wherein the user interface 1712, LED array, and the controller 1702 are integrated with the housing, wherein the touchscreen display is mounted on an external surface of the housing, and the position input 1708 may be received through a user touch contact with the touchscreen display. The touchscreen display may be a capacitive touchscreen display. The directed illumination direction may be directed to a user sleep area. In some aspects, the lighting system may further include a communication system 1718, wherein the lighting system is configured to exchange lighting data through the communication system 1718 with a second lighting system including a second communication system, a second directable lighting source, and a second controller; wherein the lighting system and the second lighting system exchange lighting data to direct the illumination direction of the directable lighting source and the second directable lighting source to a same location in the user sleep area. The lighting system may further include a sensor-based monitoring system 1714, wherein the user sleep area is monitored for a user sleep state, wherein at least one of the illumination direction or brightness may be determined at least in part by a detected change in sleep state. The sensor-based monitoring system 1714 may be at least one of a camera-based system, a radar-based system 1716, and the like. The user touch contact received through the touchscreen display may be a swiping, dragging, and the like motion of, for instance, a finger. The user touch contact may control a toggling function that toggles between a plurality of lighting settings including a utility light, a behavior light, an external light, and the like. The user touch contact received through the touchscreen display may be a dragging motion on the touchscreen display. A tapping motion may cause the controller 1702 to bring the illumination direction to a default position.

In some aspects, the techniques described herein relate to the lighting system 1700, may further include causing the controller 1702 to attenuate a brightness of the directable lighting source 1710. A field of view of the directable lighting source 1710 may be altered to execute the attenuation of the brightness of the directable lighting source 1710.

In some aspects, the techniques described herein relate to the lighting system 1700, may further include causing the controller 1702 to alter at least one of color temperature or spectral quality of the directable lighting source.

In some aspects, the techniques described herein relate to the lighting system 1700, wherein the user interface 1712 may be wirelessly connected to a mobile device, wherein the position input 1708 is received from the mobile device.

In some aspects, the techniques described herein relate to the lighting system 1700, the lighting system may further include a communication system 1718, wherein the lighting system may be configured to exchange lighting data with a second lighting system including a second communication system, a second user interface, a second directable lighting source, and a second controller; wherein the lighting system and the second lighting system exchange lighting data to coordinate to direct the illumination direction of the directable lighting source 1710 and the second directable lighting source. In some aspects, the lighting system 1700 may further include wherein the lighting system and the second lighting system exchange lighting data to coordinate to direct at least one of a brightness or color temperature of the directable lighting source 1710 and the second directable lighting source.

In some aspects, the techniques described herein relate to the lighting system 1700, wherein the directable lighting source 1710 may include a first directable lighting source and a second directable lighting source; wherein the lighting system may further include a housing, wherein the user interface 1712, first directable lighting source, and the controller 1702 may be integrated with the housing; wherein the illumination direction of the directable lighting source 1710 for the first directable lighting source and the second directable lighting source are coordinated. The coordination may direct the first directable lighting source and the second directable lighting source to a same location. The first directable lighting source may be a light emitting diode (LED) array integrated into the housing. The user interface 1712 may include a touchscreen display, wherein the directed illumination direction of the directable lighting source 1710 corresponds to the received position input 1708 received through the touchscreen display. The coordination may direct the first directable lighting source and the second directable lighting source to adjust at least one of a brightness or a color temperature.

In some aspects, the techniques described herein relate to the lighting system 1700, wherein the user interface 1712 may include at least one of a joystick controller, a light switch, touch sensor, a dimming light switch, and the like, for receiving the position input 1708, wherein the directed illumination direction of the directable lighting source 1710 corresponds to the received position input 1708 received through the at least one of the joystick controller, the light switch, or the dimming light switch. The lighting system further includes a housing, wherein the directable lighting source 1710 and the controller 1702 are integrated into the housing. The lighting system may further include a second directable lighting source, wherein the illumination direction of the directable lighting source 1710 and the second directable lighting source may be coordinated.

In some aspects, the techniques described herein relate to the lighting system 1700, wherein the lighting system may further include a second directable lighting source, wherein at least one of a brightness or a color temperature of the directable lighting source 1710 and the second directable lighting source may be coordinated.

In some aspects, the techniques described herein relate to the lighting system 1700, wherein the user interface 1712 interfaces with an application programming interface (API) to an external computing device, wherein the controller may direct the illumination direction of the directable lighting source 1710 at least in part form data received from the API.

In some aspects, the techniques described herein relate to the lighting system 1700, wherein the user interface 1712 may interface with an internet of things (IoT) device. The controller 1702 may direct at least one of the illumination direction and a brightness of the directable lighting source 1710 at least in part form data received from the IoT device. The IoT device may be an IoT dimming device, wherein the IoT dimming device controls the at least one of the illumination direction and a brightness of the directable lighting source 1710. The IoT device may be an IoT lighting device, wherein the IoT lighting device controls the at least one of the illumination direction and a brightness of the directable lighting source 1710. The IoT device may be an IoT lighting device, wherein the lighting system controls at least one of the illumination direction and a brightness of the IoT lighting device. The IoT device may be an IoT lighting device. The IoT lighting device may be one of at least an IoT light bulb, an IoT lamp, light emitting diode (LED) strip a light emitting diode (LED) array, and the like. In embodiments, the IoT lighting device may utilize a segmented light pipe for each LED or group of LEDs. The IoT lighting device may be a light emitting diode (LED) array. The LED array may further include a second controller including a second processor and a second memory, the second controller configured to store a second set of instructions that, when executed, may cause the controller to: receive the position input 1708 from the user interface; and direct the illumination direction of the LED array to correspond to the received position input 1708 from the user interface 1712.

### Sleep Restrictions

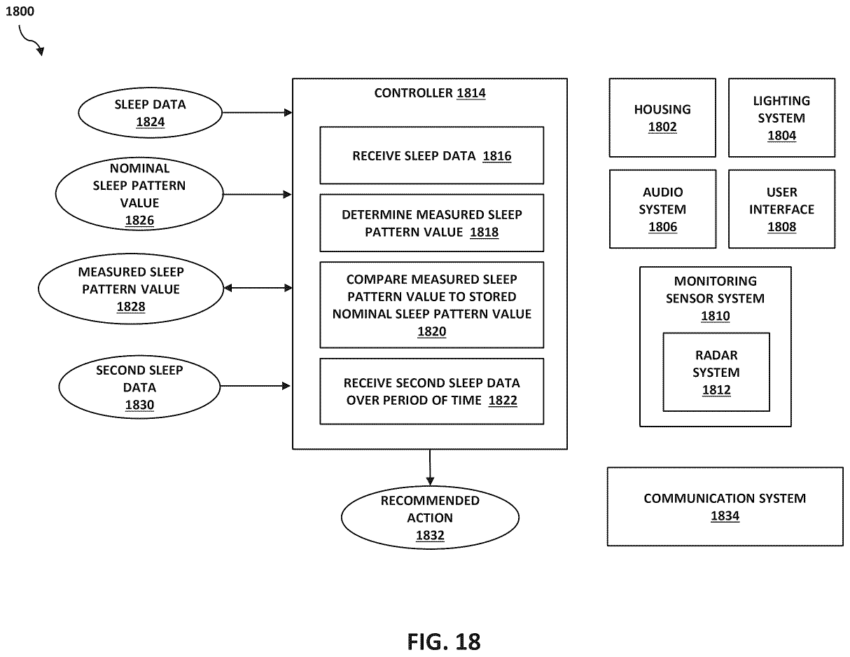

In embodiments, and referring to FIG. 18, the techniques described herein relate to a bedside sleep assistant device 1800, including: a housing 1802; a lighting system 1804 integrated with the housing 1802; an audio system 1806 integrated with the housing 1802; a user interface 1808 for receiving user commands; a monitoring sensor system 1810 for monitoring a sleep state, wherein the monitoring sensor system 1810 has an operating area including a user sleep area; and a controller 1814 including a processor and a memory, the controller 1814 configured to store a set of instructions that, when executed, may cause the controller to: receive 1816 a plurality of sleep data 1824 from the monitoring sensor system 1810 over a period of time; determine 1818 a measured sleep pattern value 1828 for a user present in the user sleep area based on the received plurality of sleep data 1824, the measured sleep pattern value 1828 a measure of sleep quality; compare 1820 the measured sleep pattern value 1828 to a stored nominal sleep pattern value 1826, the stored nominal sleep pattern value 1826 a reference measure of a healthy sleep quality; if a difference between the measured sleep pattern value 1828 and the stored nominal sleep pattern value 1826 exceeds a threshold, then provide at least one recommended action 1832 to concentrate a sleep period of the user; and receive 1822 a second plurality of sleep data 1830 over a second period of time, wherein the process to determine and compare is repeated until the difference between the measured sleep pattern value 1828 and the stored nominal sleep pattern value 1826 is changed.

In some aspects, the techniques described herein relate to the bedside sleep assistant device 1800, wherein the changed difference may be a reduced difference. In some aspects the controller 1814 may further be caused to track measured sleep pattern values to determine a sleep pattern profile, wherein the sleep pattern profile is used in part to provide the at least one recommended action 1832.

In some aspects, the techniques described herein relate to the bedside sleep assistant device 1800, the controller 1814 may be further caused to display the recommended action 1832 through the user interface 1808. In some aspects, the user interface 1808 may further include a touchscreen display, wherein the displayed recommended action may be displayed on the touchscreen display with a visual indicator that when touched confirms the recommended action 1832 be executed by the bedside sleep assistant device 1800.

In some aspects, the techniques described herein relate to the bedside sleep assistant device 1800, wherein the monitoring sensor system 1810 may include a radar system 1812, wherein the monitoring sensor system 1810 may determine the plurality of sleep data from monitoring the sleep state from at least one of movements of the user or breathing patterns of the user. At least one recommended action 1832 may be based at least in part on sleep restriction protocols. The threshold may be based at least in part on sleep restriction protocols. At least one recommended action 1832 may include a sleep schedule. At least one recommended action 1832 may include the selection of an automatic sleep assistance function. The sleep assistance function may be the setting of a lighting effect parameter. The sleep assistance function may be the setting of an audio effect parameter. The plurality of sleep data may include sleep latency data, where the monitoring sensor system measures the at least one of movements of the user or breathing patterns of the user, and the controller 1814 is caused to determine a measure of time for the user to fall asleep. The plurality of sleep data may include sleep-wake period variability data, where the monitoring sensor system 1810 measures the at least one of movements of the user or breathing patterns of the user, and the controller 1814 is caused to determine a sleep and wake time consistency measure. The plurality of sleep data may include sleep continuity data, where the monitoring sensor system 1810 measures the at least one of movements of the user or breathing patterns of the user, and the controller 1814 is caused to determine a time periods of sleep disruption measure. The plurality of sleep data may include total nighttime sleep data, where the monitoring sensor system 1810 measures the at least one of movements of the user or breathing patterns of the user, and the controller 1814 is caused to determine a measure of total hours of sleep per day for the user. The plurality of sleep data may include sleep debt data, where the monitoring sensor system 1810 measures the at least one of movements of the user or breathing patterns of the user, and the controller 1814 is caused to determine a measure of accumulated missed sleep for the user over the period of time.

In some aspects, the techniques described herein relate to the bedside sleep assistant device 1800, may further include a second bedside sleep assistant device including a second user interface, a second monitoring sensor system, and a second controller, wherein the bedside sleep assistant device 1800 and the second bedside sleep assistant device may each further include a communication system configured to exchange data between the bedside sleep assistant device and the second bedside sleep assistant device, wherein a second sleep data may include a second sleep state is received from the second bedside sleep assistant device through the communication system 1834 of the bedside sleep assistant device 1800. The controller 1814 may further use the second sleep data to determine the measured sleep pattern value 1828. In some aspects, the bedside sleep assistant device 1800 may further include transmitting sleep data to the second bedside sleep assistant device, wherein the recommended action 1832 may be a coordinated recommended action for the user being monitored by the bedside sleep assistant device 1800 and a second user being monitored by the second bedside sleep assistant device.

In some aspects, the techniques described herein relate to the bedside sleep assistant device 1800, further may include a wireless communication system 1834 in communication with an external device, wherein external sleep data may be received from the external device, wherein the controller 1814 may further use the external sleep data to determine the measured sleep pattern value 1828. The external device may be a wearable device worn by the user. The external device may be an Internet of Things (IoT) device in proximity to the user sleep area.

In some aspects, the techniques described herein relate to the bedside sleep assistant device 1800, may further include a wireless communication system in communication with an external device and the controller 1814 further caused to track measured sleep pattern values to determine a sleep pattern profile, wherein the sleep pattern profile may be transmitted to the external device. In some aspects, the bedside sleep assistant device 1800 may further include receiving external recommendation data from the external device, wherein the controller 1814 may further use the external recommendation data to provide the at least one recommended action 1832 to concentrate a sleep period of the user.

The methods and systems described herein may be deployed in part or in whole through a machine having a computer, computing device, processor, circuit, and/or server that executes computer readable instructions, program codes, instructions, and/or includes hardware configured to functionally execute one or more operations of the methods and systems disclosed herein. The terms computer, computing device, processor, circuit, and/or server, as utilized herein, should be understood broadly.

Any one or more of the terms computer, computing device, processor, circuit, and/or server include a computer of any type, capable to access instructions stored in communication thereto such as upon a non-transient computer readable medium, whereupon the computer performs operations of systems or methods described herein upon executing the instructions. In certain embodiments, such instructions themselves comprise a computer, computing device, processor, circuit, and/or server. Additionally or alternatively, a computer, computing device, processor, circuit, and/or server may be a separate hardware device, one or more computing resources distributed across hardware devices, and/or may include such aspects as logical circuits, embedded circuits, sensors, actuators, input and/or output devices, network and/or communication resources, memory resources of any type, processing resources of any type, and/or hardware devices configured to be responsive to determined conditions to functionally execute one or more operations of systems and methods herein.

Network and/or communication resources include, without limitation, local area network, wide area network, wireless, internet, or any other known communication resources and protocols. Example and non-limiting hardware, computers, computing devices, processors, circuits, and/or servers include, without limitation, a general purpose computer, a server, an embedded computer, a mobile device, a virtual machine, and/or an emulated version of one or more of these. Example and non-limiting hardware, computers, computing devices, processors, circuits, and/or servers may be physical, logical, or virtual. A computer, computing device, processor, circuit, and/or server may be: a distributed resource included as an aspect of several devices; and/or included as an interoperable set of resources to perform described functions of the computer, computing device, processor, circuit, and/or server, such that the distributed resources function together to perform the operations of the computer, computing device, processor, circuit, and/or server. In certain embodiments, each computer, computing device, processor, circuit, and/or server may be on separate hardware, and/or one or more hardware devices may include aspects of more than one computer, computing device, processor, circuit, and/or server, for example as separately executable instructions stored on the hardware device, and/or as logically partitioned aspects of a set of executable instructions, with some aspects of the hardware device comprising a part of a first computer, computing device, processor, circuit, and/or server, and some aspects of the hardware device comprising a part of a second computer, computing device, processor, circuit, and/or server.

A computer, computing device, processor, circuit, and/or server may be part of a server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more threads. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer readable instructions on a server, client, firewall, gateway, hub, router, or other such computer and/or networking hardware. The computer readable instructions may be associated with a server that may include a file server, print server, domain server, internet server, intranet server and other variants such as secondary server, host server, distributed server and the like. The server may include one or more of memories, processors, computer readable transitory and/or non-transitory media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers, and the like. Additionally, this coupling and/or connection may facilitate remote execution of instructions across the network. The networking of some or all of these devices may facilitate parallel processing of program code, instructions, and/or programs at one or more locations without deviating from the scope of the disclosure. In addition, all the devices attached to the server through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods, program code, instructions, and/or programs may be associated with a client that may include a file client, print client, domain client, internet client, intranet client and other variants such as secondary client, host client, distributed client and the like. The client may include one or more of memories, processors, computer readable transitory and/or non-transitory media, storage media, ports (physical and virtual), communication devices, and interfaces capable of accessing other clients, servers, machines, and devices through a wired or a wireless medium, and the like. The methods, program code, instructions, and/or programs as described herein and elsewhere may be executed by the client. In addition, other devices utilized for execution of methods as described in this application may be considered as a part of the infrastructure associated with the client.

The client may provide an interface to other devices including, without limitation, servers, other clients, printers, database servers, print servers, file servers, communication servers, distributed servers, and the like. Additionally, this coupling and/or connection may facilitate remote execution of methods, program code, instructions, and/or programs across the network. The networking of some or all of these devices may facilitate parallel processing of methods, program code, instructions, and/or programs at one or more locations without deviating from the scope of the disclosure. In addition, all the devices attached to the client through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules, and/or components as known in the art. The computing and/or non-computing device(s) associated with the network infrastructure may include, apart from other components, a storage medium such as flash memory, buffer, stack, RAM, ROM and the like. The methods, program code, instructions, and/or programs described herein and elsewhere may be executed by one or more of the network infrastructural elements.

The methods, program code, instructions, and/or programs described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like.

The methods, program code, instructions, and/or programs described herein and elsewhere may be implemented on or through mobile devices. The mobile devices may include navigation devices, cell phones, mobile phones, mobile personal digital assistants, laptops, palmtops, netbooks, pagers, electronic books readers, music players, and the like. These mobile devices may include, apart from other components, a storage medium such as a flash memory, buffer, RAM, ROM and one or more computing devices. The computing devices associated with mobile devices may be enabled to execute methods, program code, instructions, and/or programs stored thereon.

Alternatively, the mobile devices may be configured to execute instructions in collaboration with other devices. The mobile devices may communicate with base stations interfaced with servers and configured to execute methods, program code, instructions, and/or programs. The mobile devices may communicate on a peer to peer network, mesh network, or other communications network. The methods, program code, instructions, and/or programs may be stored on the storage medium associated with the server and executed by a computing device embedded within the server. The base station may include a computing device and a storage medium. The storage device may store methods, program code, instructions, and/or programs executed by the computing devices associated with the base station.

The methods, program code, instructions, and/or programs may be stored and/or accessed on machine readable transitory and/or non-transitory media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g., USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

Certain operations described herein include interpreting, receiving, and/or determining one or more values, parameters, inputs, data, or other information. Operations including interpreting, receiving, and/or determining any value parameter, input, data, and/or other information include, without limitation: receiving data via a user input; receiving data over a network of any type; reading a data value from a memory location in communication with the receiving device; utilizing a default value as a received data value; estimating, calculating, or deriving a data value based on other information available to the receiving device; and/or updating any of these in response to a later received data value. In certain embodiments, a data value may be received by a first operation, and later updated by a second operation, as part of the receiving a data value. For example, when communications are down, intermittent, or interrupted, a first operation to interpret, receive, and/or determine a data value may be performed, and when communications are restored an updated operation to interpret, receive, and/or determine the data value may be performed.

Certain logical groupings of operations herein, for example methods or procedures of the current disclosure, are provided to illustrate aspects of the present disclosure. Operations described herein are schematically described and/or depicted, and operations may be combined, divided, re-ordered, added, or removed in a manner consistent with the disclosure herein. It is understood that the context of an operational description may require an ordering for one or more operations, and/or an order for one or more operations may be explicitly disclosed, but the order of operations should be understood broadly, where any equivalent grouping of operations to provide an equivalent outcome of operations is specifically contemplated herein. For example, if a value is used in one operational step, the determining of the value may be required before that operational step in certain contexts (e.g. where the time delay of data for an operation to achieve a certain effect is important), but may not be required before that operation step in other contexts (e.g. where usage of the value from a previous execution cycle of the operations would be sufficient for those purposes). Accordingly, in certain embodiments an order of operations and grouping of operations as described is explicitly contemplated herein, and in certain embodiments re-ordering, subdivision, and/or different grouping of operations is explicitly contemplated herein.

The methods and systems described herein may transform physical and/or or intangible items from one state to another. The methods and systems described herein may also transform data representing physical and/or intangible items from one state to another.

The elements described and depicted herein, including in flow charts, block diagrams, and/or operational descriptions, depict and/or describe specific example arrangements of elements for purposes of illustration. However, the depicted and/or described elements, the functions thereof, and/or arrangements of these, may be implemented on machines, such as through computer executable transitory and/or non-transitory media having a processor capable of executing program instructions stored thereon, and/or as logical circuits or hardware arrangements. Example arrangements of programming instructions include at least: monolithic structure of instructions; standalone modules of instructions for elements or portions thereof, and/or as modules of instructions that employ external routines, code, services, and so forth; and/or any combination of these, and all such implementations are contemplated to be within the scope of embodiments of the present disclosure Examples of such machines include, without limitation, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipment, servers, routers and the like. Furthermore, the elements described and/or depicted herein, and/or any other logical components, may be implemented on a machine capable of executing program instructions. Thus, while the foregoing flow charts, block diagrams, and/or operational descriptions set forth functional aspects of the disclosed systems, any arrangement of program instructions implementing these functional aspects are contemplated herein. Similarly, it will be appreciated that the various steps identified and described above may be varied, and that the order of steps may be adapted to particular applications of the techniques disclosed herein.

Additionally, any steps or operations may be divided and/or combined in any manner providing similar functionality to the described operations. All such variations and modifications are contemplated in the present disclosure. The methods and/or processes described above, and steps thereof, may be implemented in hardware, program code, instructions, and/or programs or any combination of hardware and methods, program code, instructions, and/or programs suitable for a particular application. Example hardware includes a dedicated computing device or specific computing device, a particular aspect or component of a specific computing device, and/or an arrangement of hardware components and/or logical circuits to perform one or more of the operations of a method and/or system. The processes may be implemented in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and computer readable instructions, or any other machine capable of executing program instructions.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or computer-readable instructions described above. All such permutations and combinations are contemplated in embodiments of the present disclosure.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law

1. A system, comprising:
a bedside sleep assistant device, comprising:
a housing;
a lighting system integrated with the housing;
an audio system integrated with the housing;
a user interface for receiving user commands;
a monitoring sensor system for monitoring a sleep state, wherein the monitoring sensor system has an operating area including a user sleep area; and
a controller comprising a processor and a memory, the controller configured to store a set of instructions that, when executed, cause the controller to:
receive sleep data from the monitoring sensor system; and
execute a sleep assistance function that performs a sleep assist output function based on the received sleep data; and
a second bedside sleep assistant device comprising:
a second user interface,
a second monitoring sensor system, and
a second controller,
wherein the bedside sleep assistant device and the second bedside sleep assistant device each further comprise:
a communication system configured to exchange data between the bedside sleep assistant device and the second bedside sleep assistant device, wherein a second sleep data is received from the second bedside sleep assistant device through the communication system of the bedside sleep assistant device, wherein the execution of the sleep assistance function is in part determined by the received second sleep data;
wherein the monitoring sensor system further comprises a radar system, the second monitoring sensor system further comprises a second radar system, wherein the radar system detects a motion of a user in the user sleep area and the second radar system detects a motion of a second user in a second user sleep area, wherein the controller is further configured to differentiate a sleeping pattern of the user from a second sleep pattern of the second user based on the received sleep data and received second sleep data, wherein the execution of the sleep assistance function is based at least in part on the differentiation;
wherein the monitoring sensor system further comprises a microphone for receiving a sound pattern, wherein the controller is further configured to compare the received sound pattern to the motion detected by the user and the motion detected by the second user and determine if the sound pattern matches either the motion of the user or the motion of the second user, wherein the execution of the sleep assistance function is based at least in part on the determination of which user motion matches the received sound pattern.

2-60. (canceled)

61. The system of claim 1, wherein sleep assistance function is based at least in part on the determination that the motion of the user in the sleep area monitored by the bedside sleep assistant device is a match to the received sound pattern.

62. The system of claim 61, wherein the match is an indication that the user is snoring and the second user is not snoring.

63. The system of claim 62, wherein the match is determined based at least in part by a match of a frequency determined from the motion of the user and a frequency determined from the sound pattern.

64. The system of claim 62, wherein the controller is further configured to initiate a sleep routine that executes a pre-determined series of actions by the controller.

65. The system of claim 64, wherein the sleep routine is a rescue routine, wherein the pre-determined series of actions by the controller presents the actions to a user to help the user get back to sleep.

66. The system of claim 1, wherein sleep assistance function is based at least in part on the determination that the motion of the user in the sleep area monitored by the bedside sleep assistant device is determined to be a mismatch to the received sound pattern.

67. The system of claim 66, wherein the mismatch is an indication that the second user is snoring and the user is not snoring.

68-142. (canceled)
